(19) United States
(12) Patent Application Publication
(10) Pub. No.: US 2020/0023002 A1
(43) Pub. Date: Jan. 23, 2020

(54) THERAPEUTIC AGENTS AND METHODS FOR THE TREATMENT OF ACUTE RESPIRATORY DISTRESS SYNDROME AND RELATED CONDITIONS

(71) Applicant: The UAB Research Foundation, Birmingham, AL (US)

(72) Inventor: Aftab AHMAD, Hoover, AL (US)

(21) Appl. No.: 16/415,779

(22) Filed: May 17, 2019

Related U.S. Application Data
(60) Provisional application No. 62/700,537, filed on Jul. 19, 2018.

(51) Int. Cl. A61K 31/80; A61P 11/00; A61K 9/00; A61K 31/78

(52) U.S. Cl. CPC A61K 31/80; A61P 11/00; A61K 31/78; A61K 9/0053; A61K 9/0019

(57) ABSTRACT

Acute respiratory distress syndrome (ARDS) causes significant morbidity and mortality with no effective pharmacologic therapy. Protective lung ventilator strategies remain the mainstay of available treatment. New therapeutic nucleic acid scavenger compounds for the treatment of ARDS and methods of treatment of ARDS comprising administration of nucleic acid scavengers are provided.

### CROSS REFERENCE TO RELATED APPLICATIONS

This application claims benefit under 35 USC 119(e) of U.S. Ser. No. 62/700,537, filed Jul. 19, 2018.

### STATEMENT REGARDING GOVERNMENT RIGHTS

This invention was made with government support under grant number U01ES025069 awarded by the National Institute of Environmental Health Sciences. The government has certain rights in the invention

### BACKGROUND

Acute respiratory distress syndrome (ARDS) is a syndrome of acute respiratory failure caused by a variety of factors. ARDS generally presents with progressive hypoxemia, dyspnea and increased work of breathing. Patients often require mechanical ventilation and supplemental oxygen. Over the years, our understanding of ARDS has advanced significantly. However, ARDS is still associated with significant morbidity and mortality and therapeutic strategies to mitigate the foregoing have resulted in limited translational success. Part of this failure stems from heterogeneity associated with this disease.

ARDS is induced by many factors, including bacterial and viral pneumonia, sepsis, inhalation of harmful substances, head, chest or other major injury, burns, blood transfusions, near drowning, aspiration of gastric contents, pancreatitis, intravenous drug use, and abdominal trauma. Furthermore, those with a history of chronic alcoholism are at a higher risk of developing ARDS.

ARDS is often associated with fluid accumulation in the lungs. When this occurs, the elastic air sacs (alveoli) in the lungs fill with fluid and the function of the alveoli is impaired. The result is that less oxygen reaches the bloodstream, depriving organs of the oxygen required for normal function and viability. In some instances, ARDS occurs in people who are already critically ill or who have significant injuries. Severe shortness of breath, the main symptom of ARDS, usually develops within a few hours to a few days after the precipitating injury or infection.

Many patients who develop ARDS do not survive. The risk of death increases with age and severity of illness. Of the people who do survive ARDS, some recover completely while others experience lasting damage to their lungs.

There are currently no effective pharmacologic therapies for treatment or prevention of ARDS. While inhibition of fibrin formation mitigated injury in some preclinical models of ARDS, anticoagulation therapies in humans do not attenuate ARDS and may even increase mortality. Protective lung ventilator strategies remain the mainstay of available treatment options. Due to the significant morbidity and mortality associated with ARDS and the lack of effective treatment options, new therapeutic agents for the treatment of ARDS and new treatment methods for ARDS are needed.

The present disclosure addresses the unmet need in the art by providing novel therapeutic agents useful in the treatment of ARDS and methods of treatment for ARDS and conditions related thereto through the administration of such novel therapeutic agents.

### BRIEF DESCRIPTION OF THE FIGURES

FIG. 1A shows the NAS HDMBr (10 mg/kg) mitigates clinical distress scores in a CEES inhalation model of ARDS.

FIG. 1B shows the NAS EuE100 (100 mg/kg) mitigates clinical distress scores in a CEES inhalation model of ARDS.

FIG. 1C shows the NAS HDMBr (10 mg/kg) increases oxygen saturations in a CEES inhalation model of ARDS.

FIG. 1D shows the NAS EuE100 (100 mg/kg) increases oxygen saturations in a CEES inhalation model of ARDS.

FIG. 2A shows the NAS HDMBr (10 mg/kg) reverses arterial blood pH in a CEES inhalation model of ARDS.

FIG. 2B shows the NAS HDMBr (10 mg/kg) decreased PaCO₂ in a CEES inhalation model of ARDS.

FIG. 2C shows the NAS HDMBr (10 mg/kg) increased cSO₂ (calculated oxygen saturations) in a CEES inhalation model of ARDS.

FIG. 2D shows the NAS HDMBr (10 mg/kg) increased PaO₂ in a CEES inhalation model of ARDS.

FIG. 2E shows the NAS HDMBr (10 mg/kg) increased the PaO₂/FiO₂ ratio in a CEES inhalation model of ARDS.

FIG. 3A shows the NAS EuE100 (100 mg/kg) decreased PaCO₂ in a CEES inhalation model of ARDS.

FIG. 3B shows the NAS EuE100 (100 mg/kg) increased PaO₂ in an animal model of ARDS.

FIG. 3C shows the NAS EuE100 (100 mg/kg) increased cSO₂ in a CEES inhalation model of ARDS.

FIG. 3D shows the NAS EuE100 (100 mg/kg) increased the PaO₂/FiO₂ ratio in a CEES inhalation model of ARDS.

FIG. 4A shows the NAS HDMBr (10 mg/kg) decreased the steady state mRNA level of the gene encoding the pro-inflammatory cytokine IL-6 in the lungs in a CEES inhalation model of ARDS.

FIG. 4B shows the NAS HDMBr (10 mg/kg) decreased the steady state mRNA level of the gene encoding the pro-inflammatory cytokine IL1A in the lungs in a CEES inhalation model of ARDS.

FIG. 4C shows the NAS HDMBr (10 mg/kg) decreased the steady state mRNA level of the gene encoding the pro-inflammatory cytokine CXCL-1 in the lungs in a CEES inhalation model of ARDS.

FIG. 4D shows the NAS HDMBr (10 mg/kg) decreased the steady state mRNA level of the gene encoding the pro-inflammatory cytokine CCL-2 in the lungs in a CEES inhalation model of ARDS.

FIG. 4E shows the NAS HDMBr (10 mg/kg) increased the steady state mRNA level of the gene encoding the protective cytokine IL-4 in the lungs in a CEES inhalation model of ARDS.

FIG. 4F shows the NAS EuE100 (100 mg/kg) decreased the steady state mRNA level of the gene encoding the pro-inflammatory cytokine IL-6 in the lungs in a CEES inhalation model of ARDS.

FIG. 4G shows the NAS EuE100 (100 mg/kg) decreased the steady state mRNA level of the gene encoding the pro-inflammatory gene CXCL-1 in the lungs in a CEES inhalation model of ARDS.

FIG. 5A shows the NAS HDMBr (10 mg/kg) mitigated disruption of the alveolar-capillary barrier in a CEES inhalation model of ARDS as measured by total protein concentration.

FIG. 5B shows the NAS HDMBr (10 mg/kg) mitigated disruption of the alveolar-capillary barrier in a CEES inhalation model of ARDS as measured by IgM concentration.

FIG. 5C shows the NAS EuE100 (100 mg/kg) mitigated disruption of the alveolar-capillary barrier in a CEES inhalation model of ARDS.

FIG. 6A shows the NAS HDMBr (10 mg/kg) decreased the steady state mRNA level of the gene encoding the pro-coagulation factor TF in the lungs in a CEES inhalation model of ARDS.

FIG. 6B shows the NAS HDMBr (10 mg/kg) decreased the steady state mRNA level of the gene encoding the anti-fibrinolytic factor PAI-1 (plasminogen activator inhibitor-1) in the lungs in a CEES inhalation model of ARDS.

FIG. 6C shows the NAS EuE100 (100 mg/kg) decreased the steady state mRNA level of the gene encoding the pro-coagulation factor TF in the lungs in a CEES inhalation model of ARDS.

FIG. 7 shows the NAS HDMBr (10 mg/kg) reduces HMBG1 levels in a CEES inhalation model of ARDS.

FIG. 8A shows the NAS HDMBr (10 mg/kg) decreased fibrin staining in the lungs in a CEES inhalation model of ARDS.

FIG. 8B show the result of the experiment in FIG. 8A when airways were scored for fibrin casts by counting for the presence or absence of positive staining at 5× magnification. Percent of airways positive for fibrin casts were plotted.

FIG. 8C shows western blot and densitometry quantification of fibrin in BALF supernatant.

FIG. 9A shows the NAS HDMBr (10 mg/kg) decreased extracellular nucleic acid levels in the BALF supernatant in a CEES inhalation model of ARDS.

FIG. 9B shows the NAS HDMBr (10 mg/kg) decreased extracellular nucleic acid levels in the plasma in a CEES inhalation model of ARDS.

FIG. 9C show the NAS HDMBr (10 mg/kg) decreased mortality in a CEES inhalation model of ARDS.

FIG. 10A shows extracellular nucleic acid isolated from the BALF supernatant of CEES exposed rats dose-dependently increased the steady state mRNA level of the pro-inflammatory cytokine IL-6.

FIG. 10B shows isolated nucleic acid from the BALF supernatant of CEES exposed rats dose-dependently increased the steady state mRNA level of the pro-inflammatory cytokine CXCL-1.

FIG. 10C shows the NAS HDMBr (10 mg/kg) decreased steady state mRNA level of the pro-inflammatory cytokine IL-6 induced by extracellular nucleic acid.

FIG. 11 shows the NAS HDMBr (10 mg/kg) reduces lung injury in a CEES inhalation model of ARDS. Black arrow indicates airway casts. Black arrowheads show epithelial sloughing. Green arrow indicates edema and inflammatory cell infiltrations. Upper panels: 5× magnification; Lower panel: 20× magnification.

FIG. 12 shows a schematic representation of experimental schema.

FIG. 13 shows extracellular nucleic acid is increased in the BALF of rats exposed to gastric aspirate and the NAS HDMBr (10 mg/kg) reduces the levels of extracellular nucleic acid.

FIG. 14A shows the NAS HDMBr (10 mg/kg) decreased the steady state mRNA level of the gene encoding the pro-inflammatory cytokine IL-6 in the lungs in a gastric aspiration model of ARDS.

FIG. 14B shows the NAS HDMBr (10 mg/kg) decreased the steady state mRNA level of the gene encoding the pro-inflammatory cytokine IL1A in the lungs in a gastric aspiration model of ARDS.

FIG. 14C shows the NAS HDMBr (10 mg/kg) decreased the steady state mRNA level of the gene encoding the pro-inflammatory cytokine CXCL-1 in the lungs in a gastric aspiration model of ARDS.

FIG. 14D shows the NAS HDMBr (10 mg/kg) decreased the steady state mRNA level of the gene encoding the pro-inflammatory cytokine CCL-2 in the lungs in a gastric aspiration model of ARDS.

FIG. 15A shows the NAS HDMBr (10 mg/kg) mitigated disruption of the alveolar-capillary barrier in a gastric aspiration model of ARDS.

FIG. 15B shows the NAS HDMBr (10 mg/kg) mitigated disruption of the alveolar-capillary barrier in a gastric aspiration model of ARDS.

FIG. 16 shows the NAS HDMBr (10 mg/kg) reduces HMBG1 levels in a gastric aspiration model of ARDS.

FIG. 17A shows the NAS HDMBr (10 mg/kg) mitigated disruption of the alveolar-capillary barrier in GA model of ARDS as measured by total protein concentration.

FIG. 17B shows the NAS HDMBr (10 mg/kg) mitigated disruption of the alveolar-capillary barrier in a GA model of ARDS as measured by IgM concentration.

FIG. 18A shows the NAS HDMBr (10 mg/kg) decreased the steady state mRNA level of the gene encoding the pro-coagulation factor TF in the lungs in a gastric aspiration model of ARDS.

FIG. 18B shows the NAS HDMBr (10 mg/kg) decreased the steady state mRNA level of the gene encoding the anti-fibrinolytic factor PAI-1 (plasminogen activator inhibitor-1) in the lungs in a gastric aspiration model of ARDS.

FIG. 19 shows western blot and densitometry quantification of fibrin in BALF supernatant in a gastric aspiration model or ARDS.

FIG. 20 shows that extracellular nucleic acid is increased in BALF from human subjects with ARDS.

### DETAILED DESCRIPTION

### Abbreviations and Definitions

The term “nucleic acid scavenger” or “NAS” as used herein means a compound and/or composition that: i) decreases the concentration or absolute amount of extracellular nucleic acids and/or their degradation products in a subject; ii) binds to or interacts with extracellular nucleic acids to prevent or reduce the detrimental effects of extracellular nucleic acids; or iii) a combination of the foregoing. In certain embodiments, a NAS binds to or interacts with extracellular nucleic acids such that the extracellular nucleic acid is no longer able to induce its effects. In certain embodiment, a NAS may prevent, either completely or partially, an extracellular nucleic acid from binding to a cellular receptor or entering a cell. A NAS may include salts (including pharmaceutically acceptable salts), tautomeric forms, hydrates and/or solvates thereof.

In certain embodiments, a NAS is a cationic polymer. In certain embodiments, a NAS is a polyphosphate binding polymer. In certain embodiments, a NAS is hexadimethrine bromide (HDMBr; CAS No. 28728-55-4). In certain embodiments, a NAS is Eudragit E100 (EuE100; CAS No. 24938-16-7; poly(butyl methacrylate-co-(2-demethylaminoeethyl) methacrylate-co-methyl methacrylate) 1:2:1). In certain embodiments, a NAS is a β-cyclodextrin-containing polycation (CDP), In certain embodiments, a NAS is a polyamidoamine dendrimer. In certain embodiments, a NAS is a polymer comprising 1,4-diaminobutane (including, but not limited to, PAMAM G-1, PAMAM G-3 and PAMAM-G5). In certain embodiments, a NAS is a polyethylenimine polymer (PEI). In certain embodiments, a NAS is a poly(styrene-alt-maleic anhydride) co-polymer with or without PEI. Pharmaceutically acceptable salts of the foregoing are also included.

A “pharmaceutical composition” refers to a mixture of one or more of the compounds of the disclosure, with other components, such as physiologically/pharmaceutically acceptable carriers and/or excipients. The purpose of a pharmaceutical composition is to facilitate administration of a compound of the disclosure, including salts, tautomeric forms, hydrates and/or solvates to a subject (including pharmaceutically acceptable forms of the foregoing).

The term “pharmaceutically acceptable salt” is intended to include salts derived from inorganic or organic acids including, for example hydrochloric, hydrobromic, sulfuric, nitric, perchloric, phosphoric, formic, acetic, lactic, maleic, fumaric, succinic, tartaric, glycolic, salicylic, citric, methanesulfonic, benzenesulfonic, benzoic, malonic, trifluoroacetic, trichloroacetic, naphthalene-2 sulfonic and other acids. Pharmaceutically acceptable salt forms may also include forms wherein the ratio of molecules comprising the salt is not 1:1. For example, the salt may comprise more than one inorganic or organic acid molecule per molecule of base, such as two hydrochloric acid molecules per molecule of compound of the disclosure. As another example, the salt may comprise less than one inorganic or organic acid molecule per molecule of base, such as two molecules of compound of the disclosure per molecule of tartaric acid. Salts may also exist as solvates or hydrates.

The term “about” is used herein to mean approximately, roughly, around, or in the region of. When the term “about” is used in conjunction with a numerical range, it modifies that range by extending the boundaries above and below the numerical values set forth. In general, the term “about” is used herein to modify a numerical value above and below the stated value by a variance of 20 percent up or down (higher or lower).

The term an “effective amount,” “sufficient amount” or “therapeutically effective amount” as used herein is an amount of a compound that is sufficient to effect beneficial or desired results, including clinical results. As such, the effective amount may be sufficient, for example, to reduce or ameliorate the severity and/or duration of ARDS or one or more symptoms thereof, prevent the advancement of ARDS, prevent the recurrence, development, or onset of one or more symptoms associated with ARDS, enhance or otherwise improve the prophylactic or therapeutic effect(s) of another therapy for ARDS, or a combination of the foregoing. In certain embodiments, an effective amount is an amount of the compound of the disclosure that avoids or substantially attenuates undesirable side effects.

The term, “treatment” is an approach for obtaining beneficial or desired results, including clinical results. Beneficial or desired clinical results may include, but are not limited to, alleviation or amelioration of one or more symptoms or conditions, diminution of extent of disease, a stabilized (i.e., not worsening) state of disease, preventing spread of disease, delay or slowing of disease progression, amelioration or palliation of the disease state and remission (whether partial or total), whether detectable or undetectable. “Treatment” can also mean prolonging survival as compared to expected survival if not receiving treatment.

The terms “animal,” “subject” and “patient” as used herein include all members of the animal kingdom including, but not limited to, mammals, animals (e.g., cats, dogs, horses, swine, etc.) and humans. In certain embodiments, the subject is a human.

The present disclosure provides novel therapeutic agents for the treatment of ARDS and methods of treatment of ARDS and conditions related thereto through the administration of such novel therapeutic agents. More specifically, the present disclosure provides for the use of nucleic acid scavengers (NASs) as therapeutic agents for the treatment of ARDS and for the treatment of ARDS and methods of treatment of ARDS and conditions related thereto through the administration of such NASs to a subject. The use of NASs for treating ARDS and methods of treatment using NASs were not previously known in the art.

Furthermore, the present disclosure describes the role of circulating nucleic acids in causing ARDS using a rat model of aerosolized CEES (2-chloroethyl ethyl sulfide; aka: half mustard; Sulfur mustard analog) that causes moderate to severe ARDS along with significant morbidity and mortality within 12 h of exposure.

The present disclosure also comprises compositions comprising a NAS, a pharmaceutically acceptable carrier or excipient, and optionally other therapeutic agents for use in the methods of treating ARDS and conditions related thereto.

### Methods of Treatment

The present disclosure shows that NASs are effective in treating ARDS and related conditions. Mechanisms by which different insults lead to ARDS are largely unknown. The present disclosure demonstrated that NASs are effective in treating the hallmark characteristics or ARDS, such as decreasing respiratory distress, improving arterial blood gas parameters, decreasing inflammatory responses, decreasing the concentration or amount of extracellular nucleic acid, decreasing the concentration or amount of inflammatory cytokines, increasing the concentration or amount of anti-inflammatory cytokines, decreasing coagulation, increasing respiratory function, and decreasing fibrin concentration or amount. Therefore, the present disclosure shows the use of NASs represent an effective strategy in treating ARDS.

### Methods of Treating ARDS

In one embodiment, a method of treating ARDS in a subject is disclosed, such method comprising the step of administering to the subject an amount (including a therapeutically effective amount) of a NAS. In one aspect of this embodiment, ARDS is accompanied by an increase in the concentration or amount of extracellular nucleic acid.

In another embodiment, a method of treating ARDS in a subject is provided, the method comprising the step of administering to the subject an amount (including a therapeutically effective amount) of HDMBr.

In still another embodiment, a method of treating ARDS in a subject is provided, the method comprising the step of administering to the subject an amount (including a therapeutically effective amount) of EuE100.

In a further embodiment, a method of treating ARDS in a subject is disclosed, wherein the ARDS is characterized by an increase in the concentration or amount of extracellular nucleic acid, the method comprising the step of administering to the subject an amount (including a therapeutically effective amount) of a NAS.

In another embodiment, a method of treating ARDS in a subject is provided wherein the ARDS is characterized by an increase in the concentration or amount of extracellular nucleic acid, the method comprising the step of administering to the subject an amount (including a therapeutically effective amount) of HDMBr.

In still another embodiment, a method of treating ARDS in a subject is provided wherein the ARDS is characterized by an increase in the concentration or amount of extracellular nucleic acid, the method comprising the step of administering to the subject an amount (including a therapeutically effective amount) of EuE100.

### Preferred Embodiments of Methods of Treating ARDS

The following preferred embodiments are applicable to each of the methods of treating ARDS disclosed.

In certain embodiments of the methods for treating ARDS, the NAS is a cationic polymer, a polyphosphate binding polymer, a CDP, a polyamidoamine dendrimer, a polymer comprising 1,4-diaminobutane (including, but not limited to, PAMAM G-1, PAMAM G-3 and PAMAM-G5), a PEI, a poly(styrene-alt-maleic anhydride) co-polymer with or without PEI, or a combination of the foregoing. In certain embodiments of the methods for treating ARDS, the NAS is a cationic polymer. In certain embodiments of the methods for treating ARDS, the NAS is HDMBr. In certain embodiments of the methods for treating ARDS, the NAS is EuE100.

In certain embodiments of the methods for treating ARDS, the NAS is administered as a component of a pharmaceutical composition. In certain embodiments of the methods for treating ARDS, the NAS is administered, whether alone or as a part of a pharmaceutical composition, in a therapeutically effective amount.

In certain embodiments of the methods for treating ARDS, a single NAS may be administered to the subject or a combination of NASs may be administered to the subject. When a combination of NASs is administered, in certain embodiments the combination comprises HDMBr and/or EuE100. In certain embodiments of the methods for treating ARDS, the subject is determined to be in need of treatment.

In certain embodiments of the methods for treating ARDS, the ARDS is associated with or caused, at least in part, by exposure of the subject to a chemical agent. Representative chemical agents that are associated with or cause ARDS include, but are not limited to, sulfur mustards (such as, but not limited to, bis(2-chloroethyl)sulfide (CAS No. 505-60-2), 1,2-bis-(2-chloroethylthio)-ethane (CAS No. 3563-36-8), bis-(2-chloroethylthioethyl)-ether (CAS No. 63918-89-8), 2-Chloroethyl chloromethyl sulfide (CAS No. 2625-76-5), bis-(2-chloroethylthio)-methane (CAS No. 63869-13-6), bis-1,3-(2-chloroethylthio)-n-propane (CAS No. 63905-10-2), bis-1,4-(2-chloroethylthio)-n-butane (CAS No. 142868-93-7), bis-1,5-(2-chloroethylthio)-n-pentane (CAS No. 142868-94-8), bis-(2-chloroethylthiomethyl)-ether (CAS No. 63918-90-1)), half mustards (such as, but not limited to, CEES), and chlorine.

In certain embodiments of the methods for treating ARDS, the ARDS is not caused by or associated with exposure of the subject to a chemical agent. In certain embodiments of the methods for treating ARDS, the ARDS is caused, at least in part, by bacterial pneumonia, viral pneumonia, sepsis, head injury, chest injury, burns, blood transfusions, near drowning, aspiration of gastric contents, pancreatitis, intravenous drug use, abdominal trauma, or a combination of any of the foregoing.

In certain embodiments of the methods for treating ARDS, the subject is diagnosed as suffering from ARDS. In certain embodiments of the methods for treating ARDS, the subject is suspected of suffering from ARDS.

In certain embodiments of the methods for treating ARDS, the subject is a mammal. In certain embodiments of the methods for treating ARDS, the subject is a human.

In certain embodiments of the methods for treating ARDS, the NAS, either alone or as a part of a pharmaceutical composition, may be administered to the subject by any route, including, but not limited to, intravenously, intraperitoneally, parenterally, intramuscularly or orally. In some embodiments, the NAS, either alone or as a part of a pharmaceutical composition, is administered intravenously. In some embodiments, the NAS, either alone or as a part of a pharmaceutical composition, is administered intraperitoneally. In some embodiments, the NAS, either alone or as a part of a pharmaceutical composition, is administered parenterally. In some embodiments, the NAS, either alone or as a part of a pharmaceutical composition, is administered intramuscularly. In some embodiments, the NAS, either alone or as a part of a pharmaceutical composition, is administered orally.

In certain embodiments of the foregoing methods for treating ARDS, the subject is further treated with one or more additional active agents that are known for the treatment of ARDS or a condition associated with ARDS. Conditions associated with ARDS include, but are not limited to, abnormal arterial blood gas parameters (such as, but not limited to, decreased SpO₂, decreased PaO₂/FiO₂ ratio, increased PaCO₂, decreased cSO₂ and decreased PaO₂), increased inflammation or inflammatory response, increased expression of pro-inflammatory cytokines (such as but not limited to, IL-6, IL1a, CXCL-1, or CCL-2), decreased expression of anti-inflammatory cytokines (such as, but not limited to, IL-4), increased activation of the coagulation cascade, edema, increased concentration or amount of extracellular nucleic acids, or a combination of the foregoing. The one or more additional active agents and the NAS described herein can be administered together in a single composition or in separate compositions in any order, including simultaneous administration, as well as temporally spaced order of minutes to several days apart. The methods can also include more than a single administration of the one or more additional active agents and/or the NAS. The administration of the one or more additional agents and the NAS can be by the same or different routes of administration and administration of the one or more additional active agents and the NAS may occur concurrently or sequentially.

In certain embodiments of the methods for treating ARDS, the NAS is a cationic polymer and the cationic polymer is administered, either alone or as a pharmaceutical composition, in a therapeutically effective amount.

In certain embodiments of the methods for treating ARDS, the NAS is a cationic polymer and the cationic polymer is administered, either alone or as a pharmaceutical composition, in a therapeutically effective amount and the ARDS is caused, at least in part, by a chemical agent, such as, but not limited to, sulfur mustards (such as, but not limited to, bis(2-chloroethyl)sulfide (CAS No. 505-60-2), 1,2-bis-(2-chloroethylthio)-ethane (CAS No. 3563-36-8), bis-(2-chloroethylthioethyl)-ether (CAS No. 63918-89-8), 2-Chloroethyl chloromethyl sulfide (CAS No. 2625-76-5), bis-(2-chloroethylthio)-methane (CAS No. 63869-13-6), bis-1,3-(2-chloroethylthio)-n-propane (CAS No. 63905-10-2), bis-1,4-(2-chloroethylthio)-n-butane (CAS No. 142868-93-7), bis-1,5-(2-chloroethylthio)-n-pentane (CAS No. 142868-94-8), bis-(2-chloroethylthiomethyl)-ether (CAS No. 63918-90-1)), half mustards (such as, but not limited to, CEES), and chlorine.

In certain embodiments of the methods for treating ARDS, the NAS is a cationic polymer and the cationic polymer is administered, either alone or as a pharmaceutical composition, in a therapeutically effective amount and the ARDS is caused, at least in part, by bacterial pneumonia, viral pneumonia, sepsis, head injury, chest injury, burns, blood transfusions, near drowning, aspiration of gastric contents, pancreatitis, intravenous drug use, abdominal trauma, or a combination of any of the foregoing.

In certain embodiments of the methods for treating ARDS, the NAS is a cationic polymer and the cationic polymer is administered, either alone or as a pharmaceutical composition, in a therapeutically effective amount and the ARDS is not caused by a chemical agent.

In certain embodiments of the methods for treating ARDS, the NAS is HDMBr and the HDMBr is administered, either alone or as a pharmaceutical composition, in a therapeutically effective amount.

In certain embodiments of the methods for treating ARDS, the NAS is HDMBr and the HDMBr is administered, either alone or as a pharmaceutical composition, in a therapeutically effective amount and the ARDS is caused, at least in part, by a chemical agent, such as, but not limited to, sulfur mustards (such as, but not limited to, bis(2-chloroethyl)sulfide (CAS No. 505-60-2), 1,2-bis-(2-chloroethylthio)-ethane (CAS No. 3563-36-8), bis-(2-chloroethylthioethyl)-ether (CAS No. 63918-89-8), 2-Chloroethyl chloromethyl sulfide (CAS No. 2625-76-5), bis-(2-chloroethylthio)-methane (CAS No. 63869-13-6), bis-1,3-(2-chloroethylthio)-n-propane (CAS No. 63905-10-2), bis-1,4-(2-chloroethylthio)-n-butane (CAS No. 142868-93-7), bis-1,5-(2-chloroethylthio)-n-pentane (CAS No. 142868-94-8), bis-(2-chloroethylthiomethyl)-ether (CAS No. 63918-90-1)), half mustards (such as, but not limited to, CEES), and chlorine.

In certain embodiments of the methods for treating ARDS, the NAS is HDMBr and the HDMBr is administered, either alone or as a pharmaceutical composition, in a therapeutically effective amount and the ARDS is caused, at least in part, by bacterial pneumonia, viral pneumonia, sepsis, head injury, chest injury, burns, blood transfusions, near drowning, aspiration of gastric contents, pancreatitis, intravenous drug use, abdominal trauma, or a combination of any of the foregoing.

In certain embodiments of the methods for treating ARDS, the NAS is HDMBr and the HDMBr is administered, either alone or as a pharmaceutical composition, in a therapeutically effective amount and the ARDS is not caused by a chemical agent.

In certain embodiments of the methods for treating ARDS, the NAS is EuE100 and the EuE100 is administered as a part of a pharmaceutical composition in a therapeutically effective amount.

In certain embodiments of the methods for treating ARDS, the NAS is EuE100 and the EuE100 is administered, either alone or as a pharmaceutical composition, in a therapeutically effective amount and the ARDS is caused, at least in part, by a chemical agent, such as, but not limited to, sulfur mustards (such as, but not limited to, bis(2-chloroethyl)sulfide (CAS No. 505-60-2), 1,2-bis-(2-chloroethylthio)-ethane (CAS No. 3563-36-8), bis-(2-chloroethylthioethyl)-ether (CAS No. 63918-89-8), 2-Chloroethyl chloromethyl sulfide (CAS No. 2625-76-5), bis-(2-chloroethylthio)-methane (CAS No. 63869-13-6), bis-1,3-(2-chloroethylthio)-n-propane (CAS No. 63905-10-2), bis-1,4-(2-chloroethylthio)-n-butane (CAS No. 142868-93-7), Bis-1,5-(2-chloroethylthio)-n-pentane (CAS No. 142868-94-8), bis-(2-chloroethylthiomethyl)-ether (CAS No. 63918-90-1)), half mustards (such as, but not limited to, CEES), and chlorine.

In certain embodiments of the methods for treating ARDS, the NAS is EuE100 and the EuE100 is administered, either alone or as a pharmaceutical composition, in a therapeutically effective amount and the ARDS is caused, at least in part, by bacterial pneumonia, viral pneumonia, sepsis, head injury, chest injury, burns, blood transfusions, near drowning, aspiration of gastric contents, pancreatitis, intravenous drug use, abdominal trauma, or a combination of any of the foregoing.

In certain embodiments of the methods for treating ARDS, the NAS is EuE100 and the EuE100 is administered, either alone or as a pharmaceutical composition, in a therapeutically effective amount and the ARDS is not caused by a chemical agent.

Methods of Treating a Condition Associated with ARDS

In another embodiment, a method of treating a condition associated with ARDS in a subject is disclosed. Such method comprises the step of administering to the subject an amount (including a therapeutically effective amount) of a NAS. In such an embodiment, the NAS may be administered prior to a formal diagnosis of ARDS. As used herein, the term “associated with ARDS” includes a condition that is causative of ARDS and a condition that results from ARDS.

In one embodiment, the condition associated with ARDS is abnormal arterial blood gas parameters (such as, but not limited to, decreased SpO₂, decreased PaO₂/FiO₂ ratio, increased PaCO₂, decreased cSO₂ and decreased PaO₂), increased inflammation or inflammatory response, increased expression of pro-inflammatory cytokines (such as but not limited to, IL-6, IL1a, CXCL-1, or CCL-2), decreased expression of anti-inflammatory cytokines (such as, but not limited to, IL-4), increased activation of the coagulation cascade, increased pulmonary edema, increased concentration or amount of extracellular nucleic acids, or a combination of the foregoing. Any of the foregoing may be localized to the lung of a subject.

The present disclosure also shows that administration of a NAS increases oxygen saturation, decreases arterial blood pH, decreases PaCO₂, increases cSO₂, increases PaO₂, and increases the PaO₂/FiO₂ ratio in a subject suffering from ARDS.

The present disclosure provides a method for increasing oxygen saturation in a subject that is diagnosed as suffering from or suspected of suffering from ARDS, the method comprising the step of administering to the subject an amount (including a therapeutically effective amount) of a NAS.

The present disclosure provides a method for decreasing arterial blood pH in a subject that is diagnosed as suffering from or suspected of suffering from ARDS, the method comprising the step of administering to the subject an amount (including a therapeutically effective amount) of a NAS.

The present disclosure provides a method for decreasing PaCO₂ in a subject that is diagnosed as suffering from or suspected of suffering from ARDS, the method comprising the step of administering to the subject an amount (including a therapeutically effective amount) of a NAS.

The present disclosure provides a method for increasing cSO₂ in a subject that is diagnosed as suffering from or suspected of suffering from ARDS, the method comprising the step of administering to the subject an amount (including a therapeutically effective amount) of a NAS.

The present disclosure provides a method for increasing PaO₂ in a subject that is diagnosed as suffering from or suspected of suffering from ARDS, the method comprising the step of administering to the subject an amount (including a therapeutically effective amount) of a NAS.

The present disclosure provides a method for increasing the PaO₂/FiO₂ ratio in a subject that is diagnosed as suffering from or suspected of suffering from ARDS, the method comprising the step of administering to the subject an amount (including a therapeutically effective amount) of a NAS.

The present disclosure also shows that administration of a NAS decreased inflammation in the lung, decreased an inflammatory response in the lung, decreased clotting in the lung, decreased activation of the coagulation cascade in the lung, and decreased pulmonary edema in animal models of ARDS.

The present disclosure provides a method for decreasing inflammation in a subject that is diagnosed as suffering from or suspected of suffering from ARDS, the method comprising the step of administering to the subject an amount (including a therapeutically effective amount) of a NAS.

In one aspect of this embodiment, the subject is diagnosed as suffering from ARDS. In another aspect of this embodiment, the subject is suspected of suffering from ARDS. In one aspect of this embodiment, inflammation is decreased in a lung of the subject.

The present disclosure provides a method for decreasing an inflammatory response in a subject that is diagnosed as suffering from or suspected of suffering from ARDS, the method comprising the step of administering to the subject an amount (including a therapeutically effective amount) of a NAS. In one aspect of this embodiment, the subject is diagnosed as suffering from ARDS. In another aspect of this embodiment, the subject is suspected of suffering from ARDS. In one aspect of this embodiment, the inflammatory response is decreased in a lung of the subject.

The present disclosure provides a method for decreasing activation of the coagulation cascade in a subject that is diagnosed as suffering from or suspected of suffering from ARDS, the method comprising the step of administering to the subject an amount (including a therapeutically effective amount) of a NAS. In one aspect of this embodiment, the subject is diagnosed as suffering from ARDS. In another aspect of this embodiment, the subject is suspected of suffering from ARDS. In one aspect of this embodiment, the activation of the coagulation cascade is decreased in a lung of the subject.

The present disclosure provides a method for decreasing clotting in a subject that is diagnosed as suffering from or suspected of suffering from ARDS, the method comprising the step of administering to the subject an amount (including a therapeutically effective amount) of a NAS. In one aspect of this embodiment, the subject is diagnosed as suffering from ARDS. In another aspect of this embodiment, the subject is suspected of suffering from ARDS. In one aspect of this embodiment, clotting is decreased in a lung of the subject.

The present disclosure provides a method for decreasing pulmonary edema in a subject that is diagnosed as suffering from or suspected of suffering from ARDS, the method comprising the step of administering to the subject an amount (including a therapeutically effective amount) of a NAS. In one aspect of this embodiment, the subject is diagnosed as suffering from ARDS. In another aspect of this embodiment, the subject is suspected of suffering from ARDS.

The present disclosure also shows that administration of a NAS decreased the concentration and/or amount of circulating extracellular nucleic acid in animal models of ARDS. Furthermore, the present disclosure demonstrated that extracellular nucleic acid induces inflammation, coagulation and barrier dysfunction (which are characteristic of ARDS).

Therefore, in another embodiment, the present disclosure provides a method for decreasing the concentration or amount of circulating extracellular nucleic acid in a subject that is diagnosed as suffering from or suspected of suffering from ARDS. Such method comprises the step of administering to the subject an amount (including a therapeutically effective amount) of a NAS of the present disclosure. In one aspect of this embodiment, the subject is diagnosed as suffering from ARDS. In another aspect of this embodiment, the subject is suspected of suffering from ARDS. In one aspect of this embodiment, the concentration or amount of circulating extracellular nucleic acid is decreased in a lung of the subject.

The present disclosure also shows that administration of a NAS decreased the concentration and/or amount of several inflammatory cytokines in animal models of ARDS.

Therefore, in another embodiment, the present disclosure provides a method for decreasing the concentration or amount of an inflammatory cytokine in a subject that is diagnosed as suffering from or suspected of suffering from ARDS. Such method comprises the step of administering to the subject an amount (including a therapeutically effective amount) of a NAS of the present disclosure. In one aspect of this embodiment, the subject is diagnosed as suffering from ARDS. In another aspect of this embodiment, the subject is suspected of suffering from ARDS. In certain aspects of this embodiment, the inflammatory cytokine is IL-6, IL1a, CXCL-1, CCL-2, or a combination of any of the foregoing. In certain aspects of this embodiment, the mRNA levels of the inflammatory cytokine are decreased as a result of the administration. In one aspect of this embodiment, the concentration, amount, or mRNA level of an inflammatory cytokine is decreased in a lung of the subject.

The present disclosure also shows that administration of a NAS increased the concentration and/or amount of anti-inflammatory cytokines in animal models of ARDS.

Therefore, in another embodiment, the present disclosure provides a method for increasing the concentration or amount of an anti-inflammatory cytokine in a subject that is diagnosed as suffering from or suspected of suffering from ARDS. Such method comprises the step of administering to the subject an amount (including a therapeutically effective amount) of a NAS of the present disclosure. In one aspect of this embodiment, the subject is diagnosed as suffering from ARDS. In another aspect of this embodiment, the subject is suspected of suffering from ARDS. In certain aspects of this embodiment, the anti-inflammatory cytokine is IL-4. In certain aspects of this embodiment, the mRNA levels of the anti-inflammatory cytokine are increased as a result of the administration. In one aspect of this embodiment, the concentration, amount, or mRNA level of an anti-inflammatory cytokine is increased in a lung of the subject.

### Preferred Embodiments of Methods of Treating a Condition Associated with ARDS

The following preferred embodiments are applicable to each of the methods of treating a condition associated with ARDS disclosed.

In certain embodiments of the methods for treating a condition associated with ARDS, the NAS is a cationic polymer, a polyphosphate binding polymer, a CDP, a polyamidoamine dendrimer, a polymer comprising 1,4-diaminobutane (including, but not limited to, PAMAM G-1, PAMAM G-3 and PAMAM-G5), a PEI, a poly(styrene-alt-maleic anhydride) co-polymer with or without PEI, or a combination of the foregoing. In certain embodiments of the methods for treating a condition associated with ARDS, the NAS is a cationic polymer. In certain embodiments of the methods for treating a condition associated with ARDS, the NAS is HDMBr. In certain embodiments of the methods for treating a condition associated with ARDS, the NAS is EuE100.

In certain embodiments of the methods for treating a condition associated with ARDS, the NAS is administered as a component of a pharmaceutical composition. In certain embodiments of the methods for treating a condition associated with ARDS, the NAS is administered, whether alone or as a part of a pharmaceutical composition, in a therapeutically effective amount.

In certain embodiments of the methods for treating a condition associated with ARDS, a single NAS may be administered to the subject or a combination of NASs may be administered to the subject. When a combination of NASs is administered, in certain embodiments the combination comprises HDMBr and/or EuE100. In certain embodiments of the methods for treating a condition associated with ARDS, the subject is determined to be in need of treatment.

In certain embodiments of the methods for treating a condition associated with ARDS, the ARDS is associated with or caused, at least in part, by exposure of the subject to a chemical agent. Representative chemical agents that are associated with or cause ARDS include, but are not limited to, sulfur mustards (such as, but not limited to, bis(2-chloroethyl)sulfide (CAS No. 505-60-2), 1,2-bis-(2-chloroethylthio)-ethane (CAS No. 3563-36-8), bis-(2-chloroethylthioethyl)-ether (CAS No. 63918-89-8), 2-Chloroethyl chloromethyl sulfide (CAS No. 2625-76-5), bis-(2-chloroethylthio)-methane (CAS No. 63869-13-6), bis-1,3-(2-chloroethylthio)-n-propane (CAS No. 63905-10-2), bis-1,4-(2-chloroethylthio)-n-butane (CAS No. 142868-93-7), bis-1,5-(2-chloroethylthio)-n-pentane (CAS No. 142868-94-8), bis-(2-chloroethylthiomethyl)-ether (CAS No. 63918-90-1)), half mustards (such as, but not limited to, CEES), and chlorine.

In certain embodiments of the methods for treating a condition associated with ARDS, the ARDS is not caused by or associated with exposure of the subject to a chemical agent. In certain embodiments of the methods for treating a condition associated with ARDS, the ARDS is caused, at least in part, by bacterial pneumonia, viral pneumonia, sepsis, head injury, chest injury, burns, blood transfusions, near drowning, aspiration of gastric contents, pancreatitis, intravenous drug use, abdominal trauma, or a combination of any of the foregoing.

In certain embodiments of the methods for treating a condition associated with ARDS, the subject is diagnosed as suffering from ARDS. In certain embodiments of the methods for treating a condition associated with ARDS, the subject is suspected of suffering from ARDS.

In certain embodiments of the methods for treating a condition associated with ARDS, the subject is a mammal. In certain embodiments of the methods for treating a condition associated with ARDS, the subject is a human.

In certain embodiments of the methods for treating a condition associated with ARDS, the NAS, either alone or as a part of a pharmaceutical composition, may be administered to the subject by any route, including, but not limited to, intravenously, intraperitoneally, parenterally, intramuscularly or orally. In some embodiments, the NAS, either alone or as a part of a pharmaceutical composition, is administered intravenously. In some embodiments, the NAS, either alone or as a part of a pharmaceutical composition, is administered intraperitoneally. In some embodiments, the NAS, either alone or as a part of a pharmaceutical composition, is administered parenterally. In some embodiments, the NAS, either alone or as a part of a pharmaceutical composition, is administered intramuscularly. In some embodiments, the NAS, either alone or as a part of a pharmaceutical composition, is administered orally.

In certain embodiments of the methods for treating a condition associated with ARDS, the subject is further treated with one or more additional active agents that are known for the treatment of ARDS. The one or more additional active agents and the NAS described herein can be administered together in a single composition or in separate compositions in any order, including simultaneous administration, as well as temporally spaced order of minutes to several days apart. The methods can also include more than a single administration of the one or more additional active agents and/or the NAS. The administration of the one or more additional agents and the NAS can be by the same or different routes of administration and administration of the one or more additional active agents and the NAS may occur concurrently or sequentially.

In certain embodiments of the methods for treating a condition associated with ARDS, the NAS is a cationic polymer and the cationic polymer is administered, either alone or as a pharmaceutical composition, in a therapeutically effective amount.

In certain embodiments of the methods for treating a condition associated with ARDS, the NAS is a cationic polymer and the cationic polymer is administered, either alone or as a pharmaceutical composition, in a therapeutically effective amount and the ARDS is caused, at least in part, by a chemical agent, such as, but not limited to, sulfur mustards (such as, but not limited to, bis(2-chloroethyl)sulfide (CAS No. 505-60-2), 1,2-bis-(2-chloroethylthio)-ethane (CAS No. 3563-36-8), bis-(2-chloroethylthioethyl)-ether (CAS No. 63918-89-8), 2-Chloroethyl chloromethyl sulfide (CAS No. 2625-76-5), bis-(2-chloroethylthio)-methane (CAS No. 63869-13-6), bis-1,3-(2-chloroethylthio)-n-propane (CAS No. 63905-10-2), bis-1,4-(2-chloroethylthio)-n-butane (CAS No. 142868-93-7), bis-1,5-(2-chloroethylthio)-n-pentane (CAS No. 142868-94-8), bis-(2-chloroethylthiomethyl)-ether (CAS No. 63918-90-1)), half mustards (such as, but not limited to, CEES), and chlorine.

In certain embodiments of the methods for treating a condition associated with ARDS, the NAS is a cationic polymer and the cationic polymer is administered, either alone or as a pharmaceutical composition, in a therapeutically effective amount and the ARDS is caused, at least in part, by bacterial pneumonia, viral pneumonia, sepsis, head injury, chest injury, burns, blood transfusions, near drowning, aspiration of gastric contents, pancreatitis, intravenous drug use, abdominal trauma, or a combination of any of the foregoing.

In certain embodiments of the methods for treating a condition associated with ARDS, the NAS is a cationic polymer and the cationic polymer is administered, either alone or as a pharmaceutical composition, in a therapeutically effective amount and the ARDS is not caused by a chemical agent.

In certain embodiments of the methods for treating a condition associated with ARDS, the NAS is HDMBr and the HDMBr is administered, either alone or as a pharmaceutical composition, in a therapeutically effective amount.

In certain embodiments of the methods for treating a condition associated with ARDS, the NAS is HDMBr and the HDMBr is administered, either alone or as a pharmaceutical composition, in a therapeutically effective amount and the ARDS is caused, at least in part, by a chemical agent, such as, but not limited to, sulfur mustards (such as, but not limited to, bis(2-chloroethyl)sulfide (CAS No. 505-60-2), 1,2-bis-(2-chloroethylthio)-ethane (CAS No. 3563-36-8), bis-(2-chloroethylthioethyl)-ether (CAS No. 63918-89-8), 2-Chloroethyl chloromethyl sulfide (CAS No. 2625-76-5), bis-(2-chloroethylthio)-methane (CAS No. 63869-13-6), bis-1,3-(2-chloroethylthio)-n-propane (CAS No. 63905-10-2), bis-1,4-(2-chloroethylthio)-n-butane (CAS No. 142868-93-7), bis-1,5-(2-chloroethylthio)-n-pentane (CAS No. 142868-94-8), bis-(2-chloroethylthiomethyl)-ether (CAS No. 63918-90-1)), half mustards (such as, but not limited to, CEES), and chlorine.

In certain embodiments of the methods for treating a condition associated with ARDS, the NAS is HDMBr and the HDMBr is administered, either alone or as a pharmaceutical composition, in a therapeutically effective amount and the ARDS is caused, at least in part, by bacterial pneumonia, viral pneumonia, sepsis, head injury, chest injury, burns, blood transfusions, near drowning, aspiration of gastric contents, pancreatitis, intravenous drug use, abdominal trauma, or a combination of any of the foregoing.

In certain embodiments of the methods for treating a condition associated with ARDS, the NAS is HDMBr and the HDMBr is administered, either alone or as a pharmaceutical composition, in a therapeutically effective amount and the ARDS is not caused by a chemical agent.

In certain embodiments of the methods for treating a condition associated with ARDS, the NAS is EuE100 and the EuE100 is administered as a part of a pharmaceutical composition in a therapeutically effective amount.

In certain embodiments of the methods for treating a condition associated with ARDS, the NAS is EuE100 and the EuE100 is administered, either alone or as a pharmaceutical composition, in a therapeutically effective amount and the ARDS is caused, at least in part, by a chemical agent, such as, but not limited to, sulfur mustards (such as, but not limited to, bis(2-chloroethyl)sulfide (CAS No. 505-60-2), 1,2-bis-(2-chloroethylthio)-ethane (CAS No. 3563-36-8), bis-(2-chloroethylthioethyl)-ether (CAS No. 63918-89-8), 2-Chloroethyl chloromethyl sulfide (CAS No. 2625-76-5), bis-(2-chloroethylthio)-methane (CAS No. 63869-13-6), bis-1,3-(2-chloroethylthio)-n-propane (CAS No. 63905-10-2), bis-1,4-(2-chloroethylthio)-n-butane (CAS No. 142868-93-7), bis-1,5-(2-chloroethylthio)-n-pentane (CAS No. 142868-94-8), bis-(2-chloroethylthiomethyl)-ether (CAS No. 63918-90-1)), half mustards (such as, but not limited to, CEES), and chlorine.

In certain embodiments of the methods for treating a condition associated with ARDS, the NAS is EuE100 and the EuE100 is administered, either alone or as a pharmaceutical composition, in a therapeutically effective amount and the ARDS is caused, at least in part, by bacterial pneumonia, viral pneumonia, sepsis, head injury, chest injury, burns, blood transfusions, near drowning, aspiration of gastric contents, pancreatitis, intravenous drug use, abdominal trauma, or a combination of any of the foregoing.

In certain embodiments of the methods for treating a condition associated with ARDS, the NAS is EuE100 and the EuE100 is administered, either alone or as a pharmaceutical composition, in a therapeutically effective amount and the ARDS is not caused by a chemical agent.

### Preferred Nucleic Acid Scavengers

The preferred NAS are suitable for use in any of the methods of treatment disclosed herein, including methods of treating ARS and methods of treating a condition associated with ARDS. Preferred nucleic acid scavengers of the present disclosure include cationic polymers.

In one embodiment, the cationic polymer is hexadimethrine bromide (HDMBr; CAS No. 28728-55-4). In certain preferred embodiments, the weigh average molecular weight of HDMBr is from 2,000 to 20,000 g/mol, such as 2,000 to 15,000 g/mol, 2,000 to 10,000 g/mol, 2,000 to 8,000 g/mol, or 2,000 to 6,000 g/mol. In another preferred embodiment, the weigh average molecular weight of HDMBr is from 4,000 to 6,000 g/mol. In the foregoing, the weight average molecular weight is determined by size exclusion chromatography.

HDMBr is a positively charged polymer that has been used as an anti-heparin agent (heparin antagonist). It is commonly used to produce non-specific agglutination of red blood cells, attributed to neutralization of the net negative charge of the red blood cells. Because of its negative charge, heparin interferes with hexadimethrine bromide-induced red cell aggregation. It has been reported 7.5 mg of hexadimethrine bromide neutralizes 10 mg of heparin (1,000 USP units) within five minutes after intravenous injection in dogs. HDMBr has the structure of formula I below. Pharmaceutically acceptable salts of HDMBr are also included.

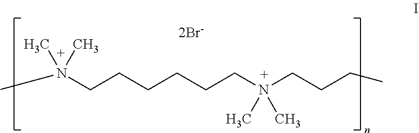

In another embodiment, the cationic polymer is Eudragit E100 (EuE100; CAS No. 24938-16-7; poly(butyl methacrylate-co-(2-demethylaminoeethyl) methacrylate-co-methyl methacrylate) 1:2:1). EuE100 is a cationic copolymer based on dimethylaminoethyl methacrylate, butyl methacrylate, and methyl methacrylate with a ratio of 2:1:1. In certain preferred embodiments, the weigh average molecular weight of EuE100 is from 10,000 to 80,000 g/mol, such as 20,000 to 70,000 g/mol, 30,000 to 60,000 g/mol, 30,000 to 40,000 g/mol, or 40,000 to 50,000 g/mol. In another preferred embodiment, the weigh average molecular weight of EuE100 is from 45,000 to 50,000 g/mol. In another preferred embodiment, the weigh average molecular weight of EuE100 is 47,000 g/mol. In the foregoing, the weight average molecular weight is determined by size exclusion chromatography.

Based on SEC method the weight average molecular weight (Mw) of is approximately 47,000 g/mol. EuE100 is commonly used in the formulation of various pharmaceuticals and as a coating for pharmaceutical dose forms to mask test. EuE100 has the structure of formula II below, wherein x is 2, y is 1 and z is 1. Pharmaceutically acceptable salts of EuE100 are also included.

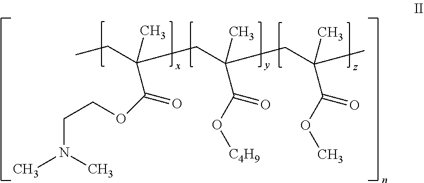

### Pharmaceutical Compositions

The NASs described herein may be formulated into pharmaceutical compositions for administration to subjects in a biologically compatible form suitable for administration in vivo. The present disclosure provides a pharmaceutical composition comprising a NAS as described herein in admixture with a pharmaceutically acceptable carrier. The pharmaceutically-acceptable carrier must be acceptable in the sense of being compatible with the other ingredients of the composition and not deleterious to the recipient thereof. The pharmaceutically-acceptable carriers employed herein may be selected from various organic or inorganic materials that are used as materials for pharmaceutical formulations and which are incorporated as analgesic agents, buffers, binders, disintegrants, diluents, emulsifiers, excipients, extenders, glidants, solubilizers, stabilizers, suspending agents, tonicity agents, vehicles and viscosity-increasing agents. Pharmaceutical additives, such as antioxidants, aromatics, colorants, flavor-improving agents, preservatives, and sweeteners, may also be added. Examples of acceptable pharmaceutical carriers include carboxymethyl cellulose, crystalline cellulose, glycerin, gum arabic, lactose, magnesium stearate, methyl cellulose, powders, saline, sodium alginate, sucrose, starch, talc and water, among others. In some embodiments, the term “pharmaceutically acceptable” means approved by a regulatory agency of the Federal or a state government or listed in the U.S. Pharmacopeia or other generally recognized pharmacopeia for use in animals, and more particularly in humans.

Often, the pharmaceutically acceptable carrier is chemically inert toward the active compounds and is non-toxic under the conditions of use. Examples of pharmaceutically acceptable carriers may include, for example, water or saline solution, polymers such as polyethylene glycol, carbohydrates and derivatives thereof, oils, fatty acids, or alcohols. In some embodiments, the carrier is saline or water. In some embodiments, the carrier is saline. In some embodiments, the carrier is water.

The pharmaceutical composition may be adapted for a particular route of administration, for example intravenous administration, intraperitoneal administration, transdermal, parenteral administration, intramuscular administration, or oral administration.

The pharmaceutical formulations of the present disclosure are prepared by methods well-known in the pharmaceutical arts. For example, the compounds of the disclosure are brought into association with a carrier and/or diluent, as a suspension or solution. Optionally, one or more accessory ingredients (e.g., buffers, flavoring agents, surface active agents, and the like) also are added. The choice of carrier is determined by the solubility and chemical nature of the compounds, chosen route of administration and standard pharmaceutical practice. In some embodiments, the formulation comprises a compound of the disclosure and water. In some embodiments, the formulation comprises a compound of the disclosure and saline.

Additionally, the compounds of the disclosure are administered to a subject, such as a human or animal subject, by known procedures including, without limitation, oral administration, sublingual or buccal administration, parenteral administration, transdermal administration, via inhalation or intranasally, vaginally, rectally, and intramuscularly. The compounds of the disclosure may be administered parenterally, by epifascial, intracapsular, intracranial, intracutaneous, intrathecal, intramuscular, intraorbital, intraperitoneal, intraspinal, intrasternal, intravascular, intravenous, parenchymatous, subcutaneous or sublingual injection, or by way of catheter. In some embodiments, the compounds of the disclosure are administered to the subject by way of intramuscular delivery. In some embodiments, the compounds of the disclosure are administered to the subject by way of intraperitoneal delivery. In some embodiments, the compounds of the disclosure are administered to the subject by way of intravenous delivery. In some embodiments, the compounds of the disclosure are administered orally. In certain embodiments, the compounds of the disclosure are administered by bolus administration, for example an IV or IM bolus administration.

For oral administration, a formulation of the compound of the disclosure may be presented as capsules, tablets, powders, granules, or as a suspension or solution. Capsule formulations may be gelatin, soft-gel or solid. Tablets and capsule formulations may further contain one or more adjuvants, binders, diluents, disintegrants, excipients, fillers, or lubricants, each of which are known in the art. Examples of such include carbohydrates such as lactose or sucrose, dibasic calcium phosphate anhydrous, corn starch, mannitol, xylitol, cellulose or derivatives thereof, microcrystalline cellulose, gelatin, stearates, silicon dioxide, talc, sodium starch glycolate, acacia, flavoring agents, preservatives, buffering agents, disintegrants, and colorants. Orally administered compositions may contain one or more optional agents such as, but not limited to, sweetening agents such as fructose, aspartame or saccharin; flavoring agents such as peppermint, oil of wintergreen, or cherry; coloring agents; and preservative agents, to provide a pharmaceutically palatable preparation.

For parenteral administration (i.e., administration by injection through a route other than the alimentary canal), the compounds of the disclosure may be combined with a sterile aqueous solution that is isotonic with the blood of the subject. Such a formulation is prepared by dissolving a solid active ingredient in water containing physiologically-compatible substances, such as sodium chloride, glycine and the like, and having a buffered pH compatible with physiological conditions, so as to produce an aqueous solution, then rendering said solution sterile. The formulation may be presented in unit or multi-dose containers, such as sealed ampules or vials.

The formulation may be delivered by any mode of injection, including, without limitation, epifascial, intracapsular, intracranial, intracutaneous, intrathecal, intramuscular, intraorbital, intraperitoneal, intraspinal, intrasternal, intravascular, intravenous, parenchymatous, subcutaneous, or sublingual or by way of catheter into the subject's body.

Parenteral administration includes aqueous and non-aqueous based solutions. Examples of which include, for example, water, saline, aqueous sugar or sugar alcohol solutions, alcoholic (such as ethyl alcohol, isopropanol, glycols), ethers, oils, glycerides, fatty acids, and fatty acid esters. In some embodiments, water is used for parenteral administration. In some embodiments, saline is used for parenteral administration. Oils for parenteral injection include animal, vegetable, synthetic or petroleum based oils. Examples of sugars for solution include sucrose, lactose, dextrose, mannose, and the like. Examples of oils include mineral oil, petrolatum, soybean, corn, cottonseed, peanut, and the like. Examples of fatty acids and esters include oleic acid, myristic acid, stearic acid, isostearic acid, and esters thereof.

For transdermal administration, the compounds of the disclosure are combined with skin penetration enhancers, such as propylene glycol, polyethylene glycol, isopropanol, ethanol, oleic acid, N-methylpyrrolidone and the like, which increase the permeability of the skin to the compounds of the disclosure and permit the compounds to penetrate through the skin and into the bloodstream. The compound/enhancer compositions also may be further combined with a polymeric substance, such as ethylcellulose, hydroxypropyl cellulose, ethylene/vinylacetate, polyvinyl pyrrolidone, and the like, to provide the composition in gel form, which are dissolved in a solvent, such as methylene chloride, evaporated to the desired viscosity and then applied to backing material to provide a patch.

In some embodiments, the compounds of the disclosure are in unit dose form such as a tablet, capsule or single-dose vial. Suitable unit doses, i.e., an effective amount, may be determined during clinical trials designed appropriately for each of the conditions for which administration of a chosen compound is indicated and will, of course, vary depending on the desired clinical endpoint.

The present disclosure also provides articles of manufacture for treating ARDS and conditions related thereto in a subject. The articles of manufacture comprise a compound of the disclosure or a pharmaceutical composition comprising a compound of the disclosure, optionally further containing at least one additional active agent, as described herein. For example, the present disclosure provides for the use of a NAS in the manufacture of a medicament for the treatment of ARDS and conditions related thereto in a subject. The present disclosure also provides for the use of a NAS in the treatment of ARDS and conditions related thereto in a subject. The articles of manufacture may be packaged with indications for various disorders that the pharmaceutical compositions are capable of treating. For example, the articles of manufacture may comprise a unit dose of a compound of the disclosure that is capable of treating or preventing ARDS and conditions related thereto in a subject, and an indication that the unit dose is capable of treating ARDS and conditions related thereto in a subject.

### Dosage and Administration

In accordance with the methods of the present disclosure, a NAS of the disclosure is administered to the subject (or are contacted with cells of the subject) in a therapeutically effective amount. This amount may be determined by the skilled artisan, based upon known procedures, including analysis of titration curves established in vivo and methods and assays disclosed herein.

In certain embodiments, the therapeutically effective amount of a NAS as described herein ranges from about 0.01 mg/kg/day to about 500 mg/kg/day. In certain embodiments, the therapeutically effective amount ranges from about 0.01 mg/kg/day to about 400 mg/kg/day. In certain embodiments, the therapeutically effective amount ranges from about 0.01 mg/kg/day to about 300 mg/kg/day. In certain embodiments, the therapeutically effective amount ranges from about 0.01 mg/kg/day to about 200 mg/kg/day. In certain embodiments, the therapeutically effective amount ranges from about 0.01 mg/kg/day to about 100 mg/kg/day. In certain embodiments, the therapeutically effective amount ranges from about 0.01 mg/kg/day to about 50 mg/kg/day. In certain embodiments, the therapeutically effective amount ranges from about 0.01 mg/kg/day to about 25 mg/kg/day. In certain embodiments, the therapeutically effective amount ranges from about 0.01 mg/kg/day to about 20 mg/kg/day. In certain embodiments, the therapeutically effective amount ranges from about 0.01 mg/kg/day to about 15 mg/kg/day. In certain embodiments, the therapeutically effective amount ranges from about 0.01 mg/kg/day to about 10 mg/kg/day. In certain embodiments, the therapeutically effective amount ranges from about 0.01 mg/kg/day to about 5 mg/kg/day. In certain embodiments, the therapeutically effective amount ranges from about 0.01 mg/kg/day to about 2.5 mg/kg/day. In some embodiments, the therapeutically effective amount ranges from about 5 mg/kg/day to about 100 mg/kg/day. In some embodiments, the effective amount ranges from about 5 mg/kg/day to about 50 mg/kg/day. In some embodiments, the therapeutically effective amount ranges from about 5 mg/kg/day to about 30 mg/kg/day. In some embodiments, the therapeutically effective amount ranges from about 5 mg/kg/day to about 10 mg/kg/day.

In some embodiments, the therapeutically effective amount of a NAS as described herein is between about 0.1 mg/kg/day and about 50 mg/kg/day. In some embodiments, the therapeutically effective amount is between about 0.1 mg/kg/day and about 40 mg/kg/day. In some embodiments, the therapeutically effective amount is between about 0.1 mg/kg/day and about 30 mg/kg/day. In some embodiments, the therapeutically effective amount is between about 0.1 mg/kg/day and about 20 mg/kg/day. In some embodiments, the therapeutically effective amount is between about 0.1 mg/kg/day and about 10 mg/kg/day. In some embodiments, the therapeutically effective amount is between about 0.1 mg/kg/day and about 5 mg/kg/day.

In certain embodiments, the therapeutically effective amount is administered in one or more doses according to a course of treatment (where a dose refers to an amount of a NAS administered in a single day). In certain embodiments, the dose is administered q.d. (1 time/administration per day). In certain embodiments, the dose is administered b.i.d. (2 times/administrations per day; for example, one-half of the therapeutically effective amount in two administrations a day). In certain embodiments, the dose is administered t.i.d. (three times/administrations per day; for example, one-third of the therapeutically effective amount in three administrations a day). When a dose is divided into multiple administrations per day, the dose may be divided equally or the dose may be divided unequally at each administration. Any given dose may be delivered in a single dosage form or more than one dosage form (for example, a tablet).

### Kits

Also provided by the present disclosure are kits. Typically, a kit includes a NAS of the present disclosure. In certain embodiments, a kit can include a delivery system for the NAS, and directions for use of the kit (e.g., instructions for treating a subject). In certain embodiments, a kit can include the NAS and an additional active agent useful in treating ARDS or a condition related thereto. In some embodiments, the NAS is a cationic polymer. In some embodiments, the NAS is HDMBr. In some embodiments, the NAS is EuE100. In certain embodiment, the condition associated with ARDS is abnormal arterial blood gas parameters (such as, but not limited to, decreased SpO₂, decreased PaO₂/FiO₂ ratio, increased PaCO₂, decreased cSO₂ and decreased PaO₂), increased inflammation or inflammatory response, increased expression of pro-inflammatory cytokines (such as but not limited to, IL-6, IL1a, CXCL-1, or CCL-2), decreased expression of anti-inflammatory cytokines (such as, but not limited to, IL-4), increased activation of the coagulation cascade, increased pulmonary edema, increased concentration or amount of extracellular nucleic acids, or a combination of the foregoing. Any of the foregoing may be localized to the lung of a subject.

### Methods

The methods are further described in “Neutralization of extracellular nucleic acids rescues rats from chemical-induced ARDS” (American Journal of Respiratory and Critical Care Medicine, Mariappan, N. et al., submitted for publication), which reference is incorporated herein in its entirety, except for any definitions, subject matter disclaimers or disavowals, and except to the extent that the incorporated material is inconsistent with the express disclosure herein, in which case the language in this disclosure controls.

### Animal Models of ARDS

The animal models used in the present disclosure, the CEES inhalation and GA models, cause acute respiratory failure that captures multiple pathological features of ARDS observed in humans. Hypercoagulation, an exacerbated inflammatory response, hypoxemia and disruption of the alveolar capillary barrier are some of the cardinal features of ARDS in humans that are also manifested in CEES- and GA-induced lung injuries

### Aerosolized CEES Inhalation Model

The model used in Examples 1 to 11 is an aerosolized CEES inhalation in rats. CEES is an analog of sulfur mustard that causes moderate to severe ARDS along with significant morbidity and mortality within 12 h of exposure. This model system mimics many of the pathophysiologic features characteristic of ARDS (3, 4). The present study investigates the use of nucleic acid scavengers as therapeutic agents and the role of circulating nucleic acids in ARDS injury and progression.

Sulfur mustard (SM; bis(2-chloroethyl ethyl)sulfide) and its less potent analog CEES are highly reactive alkylating and vesicating agent (5). It has reemerged as a threat and war agent that can cause large scale morbidity and mortality. Our understanding of the pathophysiology is derived from human and animal exposures to SM or CEES. Most acute inhalation exposure affects both the upper and lower airways (6, 7) causing injury, impaired gas exchange and epithelial sloughing (8, 9). Acute exposures to SM/CEES also cause severe lung inflammation, airway hyperreactivity, pulmonary edema and activation of the coagulation cascade resulting in formation of fibrin rich bronchial plugs causing airway obstruction leading to death (10, 11). Increased morbidity and mortality is also attributed to the fact that exposure to SM/CEES causes multiple organ failure (12-15). In some preclinical models of ALI/ARDS, inhibition of fibrin formation mitigated injury (16, 17). Despite these promising studies there is little evidence that anticoagulation therapies attenuates ARDS in humans and in some cases there was even increases in mortality (18).

After acute exposure to SM/CEES, several factors may activate the coagulation cascade and promote clot formation in the bloodstream and within airspaces of the lung (5). Growing evidence indicates that circulating extracellular nucleic acids (eNA) can activate the coagulation cascade and also prevent fibrinolysis thereby increasing the stability of fibrin clots (19, 20). Thus, eNAs can affect multiple steps of the coagulation pathway. Extracellular nucleic acids are also increasingly being recognized as important mediators of inflammation and injury (21-24). These nucleic acids include amongst other, ribosomal RNA, miRNA, tRNA, mRNA, nuclear and mitochondrial DNA (25). Nucleic acids released from the cells as a result of injury or through the normal apoptotic process can activate multiple signaling pathways (26, 27). These nucleic acids can also serve as diagnostic and prognostic markers of various diseases (28). Nucleic acids released from cell can also stimulate the pattern-recognition receptors and are potent activators of the inflammatory pathway (29). They are therefore unique molecules that can accelerate the development of inflammatory response and activate the coagulation cascade. Since both the coagulation and inflammatory pathways are activated in CEES/SM-induced injury, we hypothesized that extracellular nucleic acids play an important mechanistic role in the pathogenesis of this injury.

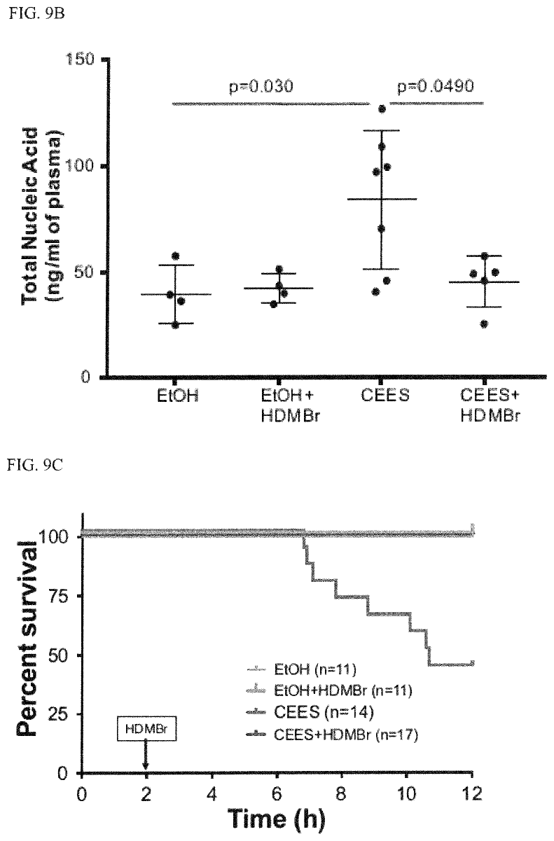

Adult male (275 to 300-g) Sprague-Dawley rats from Envigo Co., (Indianapolis, Ind.) were used. Animals were provided food and water ad libitum and maintained at 25° C. in a 12-hour light/dark cycle room. Animals were randomly assigned to one of four groups for the HDMBr study: 1) ethanol (aerosolized ethanol as diluent for CEES); 2) ethanol+HDMBr (aerosolized ethanol+HDMBr); 3) CEES (10% aerosolized CEES in ethanol); or 4) CEES+HDMBr (10% aerosolized CEES in ethanol+HDMBr). HDMBr was administered intraperitoneally at 2 hours post CEES administration at 10 mg/kg (in PBS); control animals were administered PBS intraperitoneally (FIG. 12).

Animals were randomly assigned to one of three groups for the EuE100 study: Ethanol (aerosolized ethanol as diluent for CEES), CEES (10% aerosolized CEES in ethanol) or CEES+EuE100 (10% aerosolized CEES in ethanol+EuE100). EuE100 was administered intraperitoneally at 2 and 6 hours post CEES administration at 100 mg/kg (in PBS); control animals were administered PBS intraperitoneally (FIG. 12).

The exposure system (nose-only inhalation, CH Technologies, NJ) has been described previously with minor modification (31). Briefly, rats were anesthetized with a mixture of ketamine (75 mg/kg), xylazine (7.5 mg/kg), and acepromazine (1.5 mg/kg). They were then placed in sealed restraint tubes for containment and attached to the exposure chamber operated under negative pressure. A solution of CEES (10% in ethanol) was injected through a syringe pump and the contents aerosolized for 15 minutes using a bioaerosol nebulizing generator (BANG). After 15 minutes of exposure, rats were returned to their cages and observed until they had fully recovered from anesthesia. At the indicated time points after CEES administration, rats were administered doses of NAS as described.

Animals were monitored continuously and mean clinical distress scores were recorded. Heart rates and percent oxygen saturations were also recorded every 2 hours using a MouseOx pulseoximeter. Survival times were measured for each animal as the time from CEES exposure until the animals died or until they met the euthanasia criterion and were euthanized as described previously (33). After 12 h of CEES exposure, animals were euthanized and bronchoalveolar lavage fluid (BALF) and blood were collected from the lung and descending aorta, respectively. Blood samples form the descending aorta were used for measurement of arterial blood gas as described herein. Samples were also collected from animals that met euthanasia criterion earlier. Lungs were perfused through the pulmonary artery and tissues were snap-frozen in liquid nitrogen. Separate animals were used for fixed tissues. Lungs were inflation fixed in 4% paraformaldehyde in phosphate-buffered saline (PBS) with a pressure resembling 20 cm of water.

### Gastric Aspiration Animal Model

Gastric aspiration caused by gastro-oesophageal reflux is a condition in which the gastric contents leak into the respiratory tract. Such infiltration of the gastric contents into the respiratory tract can lead to ARDS.

In order to study the effects of gastro-oesophageal reflux in ARDS, a gastric aspiration model of rat was created. Lung injury in adult rats following the aspiration of dilute hydrochloric acid in this model is characterized by a bi-phasic response comprising an early insult that is characterized by stimulation of capsaicin-sensitive neurons and direct caustic actions of low pH on airway epithelium followed by an acute neutrophilic inflammatory response at 4-6 hr. These pathogenic mechanisms lead to the loss of pulmonary microvascular integrity and extravasation of fluid and protein into the airways and alveoli. In addition to mechanically increasing the work of breathing by increasing airway resistance and inhibiting the diffusion of oxygen, edema fluid contains plasma proteins and other substances that can directly interfere with the function of alveolar surfactant

In this model, HCl (0.5N) is mixed with gastric contents with a pH on 1.2 to simulate gastric aspirate (GA). The GA was instilled in adult male Sprague-Dawley rats (175-200 g) through 20-gauge endotracheal tube. As control, rats received the same volume of saline via intratracheal instillation. Animals were provided food and water ad libitum and maintained at 25° C. in a 12-hour light/dark cycle room. Animals were randomly assigned to one of four groups as follows: 1) saline (0.9%); 2) saline+HDMBr, 3) GA; or 4) GA+HDMBr. Two hours after saline or GA administration, HDMBr (10 mg/kg) or saline was administered intraperitoneally. At 6 hours post administration of GA or saline, the animals were euthanized and bronchoalveolar lavage fluid (BALF) and blood were collected from the lung and descending aorta, respectively. Lungs were also collected and used for RNA isolation. Separate animals were used for preparation of fixed tissues.

### Chemicals

2-Chloroethyl ethyl sulfide (CEES, C₄H₉CIS) and hexadimethrine bromide (HDMBr) were purchased from Sigma-Aldrich Chemical Co (St. Louis, Mo.). Antibodies for HMGB1 and anti-human fibrinogen were obtained from Abcam and DAKO respectively. EuE100 was obtained from Evonik (Essen, Germany).

### Animals

All experiments involving animals were conducted according to protocols approved by the Institutional Animal Care and Use Committee of the University of Alabama at Birmingham.

### Clinical Distress Scoring

Respiratory quality, stridor and activity levels were assessed for every 2 h after CEES exposure with each variable scored at 0-3, with higher numbers indicating the greatest distress. The three category scores were added to obtain a cumulative score (maximum 9). Criteria for early euthanasia were oxygen saturation less than 70% and plus respiratory distress score of 7 or greater, as described previously (10) and as shown below.

| Score | Stridor | Respiratory Quality | Activity |
|---|---|---|---|
| 0 | Normal | Normal breathing | Normal |
| 1 | Mild stridor w/activity only, at | Mild abdominal breathing + | Mildly depressed activity |
| | rest no mouth breathing/ | tachypnea (>100) | |
| | gasping | | |
| 2 | Mild to mod. stridor at rest, | Mod. Abdominal breathing | Mod. depressed activity |
| | head bobbing present at rest | (abdomen sucked in with breaths), | (moves some with |
| | (gasping) | RR between 60-100, possibly with | stimulation) |
| | | mild gasping | |
| 3 | Severe stridor, severe head- | Severe gasping, low respiratory | Obtunded, no movement w/ |
| | bobbing | rate (<60) | stimulation, or severe |
| | | | agitation w/stimulation |

### Pulse Oximetry

Oxygen saturation and heart rate were monitored using the MouseOX oximeter (Starr Life Science, Pittsburgh, Pa.) in un-anesthetized rats before CEES exposure, and every 2 h following CEES exposure. Three measurements per time point were taken.

### Arterial Blood Gas Measurements

To assess pulmonary gas exchange, blood gas analyses were performed by obtaining blood from the descending aorta. Heparinized whole blood was analyzed using calibrated test cards (EPOC-BGEM) and a Heska EPOC blood gas and electrolyte analyzer (Loveland, Colo.).

### Protein and IgM Measurement

BALF supernatant was collected as previously described (10, 31). To obtain BAL fluid, the lungs were lavaged 2 times with 5 ml of PBS solution. Collected lavage fluid was centrifuged at 800 g for 10 min; aliquots of supernatants were frozen at −80° C. until further measurements. Total protein concentration was measured in the BALF supernatants using the Bio-Rad DC method with bovine serum albumin (BSA) as a standard. All samples were assayed in duplicate. IgM was measured using a standard ELISA kit (Bethyl Lab Inc, Montgomery, Tex.).

### Differential Cell Counts

BALF was centrifuged, and the pellet was re-suspended in 2 ml of PBS. From this, 100 ul of BALF was centrifuged in a Cytospin (Shandon Inc.). Cells on the slide were then air-dried and stained to count neutrophils and macrophages using the Hema 3 differential stain (Fisher Diagnostics, Middle town, VA).

### Measurement of Blood Parameters

Blood samples were collected at euthanasia for analysis of complete blood count (CBC). An aliquot of whole blood was transferred to EDTA-coated vials and analyzed using hematology analyzer (HEMAVET 950 FS; Drew Scientific, Dallas, Tex.).

### Isolation of Extracellular Nucleic Acid and In Vitro Studies

The total circulating nucleic acids were isolated from 200 ul of plasma (platelet free) and BALF using a commercially available kit (Cat #56300, Norgen BIOTEK CORP., Ontario, CA) according to the manufacturer's instructions. Nucleic acid concentration was measured using a microvolume spectrophotometer (DeNovix Inc. Wilmington, Del., USA). The effect of CEES plasma-derived extracellular nucleic acid was tested on lung epithelial cells in presence or absence of HDMBr. Extracellular nucleic acid was preincubated with HDMBr (10 ug/ml) or vehicle for 30 min and added to human bronchial epithelial cells (16HBE). After 6 h incubation in a cell culture incubator, cells were harvested, RNA isolated and Realtime RT PCR was carried out using specific primers and probes.

### Gene Expression

Total RNA was extracted using an RNeasy Mini Kit (Cat #74106, Qiagen Co, USA). RNA quality was assessed using Agilent Bioanalyser 2100 (Agilent). For quantitative RT-PCR, first-strand cDNA was reverse transcribed from 1 μg of total RNA using the iScript reverse transcription super mix (Cat #1708840, Bio-Rad laboratories Inc.). For each sample, 50 ng cDNA (total RNA equivalent) was amplified in a 25 μl reaction volume using the Bio-Rad CFX 96 real-time PCR machine (Bio-Rad laboratories Inc.) as per the instructions specified by the manufacturer. Taqman primers specific for IL1A (Rn00566700_m1), IL-6 (Rn01410330_m1), IL-4 (Rn01456866_m1), CCL-2 (Rn00580555), CXCL-1 (Rn00578225_m1), PAI-1 (Rn01481341_m1), and TF (Rn00564925_m1) were used for gene expression analysis. Results were normalized to β-actin (Rn00667869_m1) or 18S rRNA (4310893-E) that were used as housekeeping genes and calculated as a ratio of gene expression to the expression of the reference gene, β-actin or 18S. All primers/probes sets for cytokines/chemokines and coagulation genes were procured from Applied Biosystems.

### Histology and Immunohistochemistry

The lung was inflated for 30 minutes at a constant hydrostatic pressure of 20 cm with 4% buffered formalin and immersed in the same solution for 24 h. The fixed lung was trimmed, embedded in paraffin, and cut into 5 um sections. The sections were stained with hematoxylin and eosin (H&E) for morphological examination.

For immunohistochemistry, lung sections were processed and stained using specific antibodies. Immunostaining for fibrin was performed using a polyclonal rabbit anti-human fibrinogen (DAKO) at a 1:2000 dilution for 60-min. Rabbit IgG control (DAKO) was used at the same specifications. The stains were developed using a peroxidase-based Envision detection system (DAKO; Carpinteria, Calif.). The counterstaining was performed using hematoxylin.

### Western Blotting

Western blotting for HMGB1 and fibrin were carried out in bronchoalveolar lavage fluid. Forty microliters of BALF was resolved in a 4-20% SDS-PAGE gradient gel, transferred to a nitrocellulose membrane and probed with antibodies against HMGB1 and Fibrin(ogen). Blots were developed using a chemiluminescent substrate and imaged in a Chemidoc Imager (BioRAD). The bands were quantitated and plotted.

### Statistical Analysis

Prism 7.0 software (GraphPad Prism, La Jolla, Calif.) was used, with one-way analysis of variance (ANOVA) followed by Bonferroni's multiple comparisons test, unless otherwise indicated. Data reported are mean values with standard error of the mean (SEM). A p value of <0.05 was considered significant.

### EXAMPLES

### Example 1—Treatment with a Nucleic Acid Scavenger Improves Clinical Distress Scores and Oxygen Saturations in the CEES Inhalation Model of Acute Respiratory Distress Syndrome

ARDS impacts physical activity, respiratory quality and stridor, which can be cumulatively expressed numerically as clinical distress scores (a scale of 1-9) (33). Clinical distress scores are commonly used to measure the severity of ARDS. The higher the clinical distress score, the worse the disease. In order to investigate the impact of NASs on clinical distress scores and oxygen saturations in an in vivo model of ARDS, Sprague Dawley rats were exposed to aerosolized CEES and NASs (HDMBr and EuE100) as described in the Methods section herein.

Clinical distress scores were recorded and plotted over time. Noninvasively acquired oxygen saturations (SpO₂) were measured using a MouseOx pulseoximeter and plotted over time. Data were analyzed by one-way ANOVA followed by Bonferroni multiple comparison test. Values represent mean±SEM. A * indicates p<0.05 relative to the CEES exposed group.

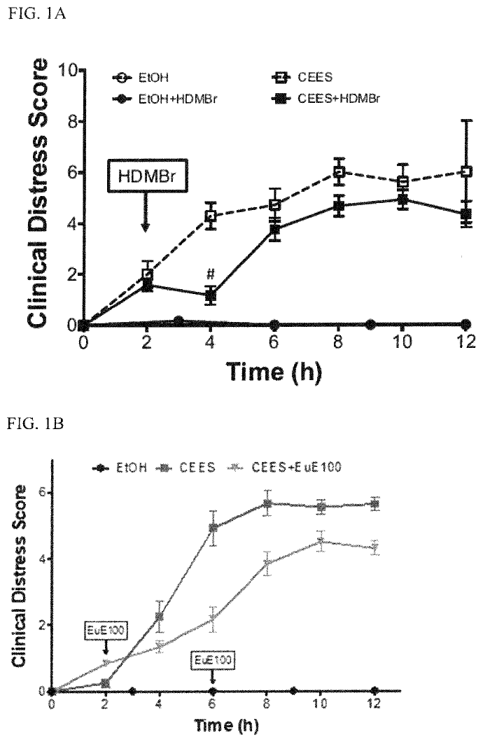

Exposure to CEES resulted in a progressive increase in clinical distress scores in treated animals. Control groups showed no increase in clinical distress (FIGS. 1A and 1B). Treatment with the NASs HDMBr at a concentration of 10 mg/kg (FIG. 1A) and EuE100 at a concentration of 100 mg/kg (FIG. 1B) decreased clinical distress scores in CEES-exposed rats.

Non-invasive pulse oximetry can be employed to effectively assess oxygenation criterion in ARDS longitudinally (34). Following exposure of rats to aerosolized CEES, oxygen saturation (SpO₂) was measured every 2 hours until study end point or until the animal died. Compared to the controls groups, exposure to aerosolized CEES caused progressive decrease in SpO₂ (FIGS. 1C and 1D). Treatment with the NASs HDMBr at a concentration of 10 mg/kg (FIG. 1C) and EuE100 at a concentration of 100 mg/kg (FIG. 1D) improved oxygenation in CEES-exposed rats.

### Example 2—Treatment with a Nucleic Acid Scavenger Improves Arterial Blood Gas (ABG) Measurements in the CEES Inhalation of Acute Respiratory Distress Syndrome

Arterial blood gas measurements are routine clinical assessments used to evaluate extent of lung injury, impairment in gas exchange and subsequent hypoxemia and are routinely carried out in patients with ARDS. In ARDS patients, there is increased blood CO₂ levels and decreased blood oxygen levels indicative of respiratory dysfunction. The effect of NASs on ABG measurements was determined in an in vivo model of ARDS. The same animal model system described in Example 1 was used in this experiment.

In this animal model system, CEES exposure causes respiratory acidosis (33). Various ABG parameters, including arterial blood pH, partial pressure of arterial carbon dioxide (PaCO₂), bicarbonate levels (HCO₃⁻), calculated oxygen saturation (cSO₂), partial pressure of arterial oxygen (PaO₂), and PaO₂/FiO₂ ratio, were measured after exposure to aerosolized CEES and/or NASs. At the end of study (12 h), blood was collected from the descending aorta and analyzed for ABGs using the EPOC-Vet Blood Analysis System. Data were compared in rats given CEES versus CEES+HDMBr and analyzed by one-way ANOVA followed by Bonferroni multiple comparison test. Values represent mean±SEM; p-values for various group comparisons are provided.

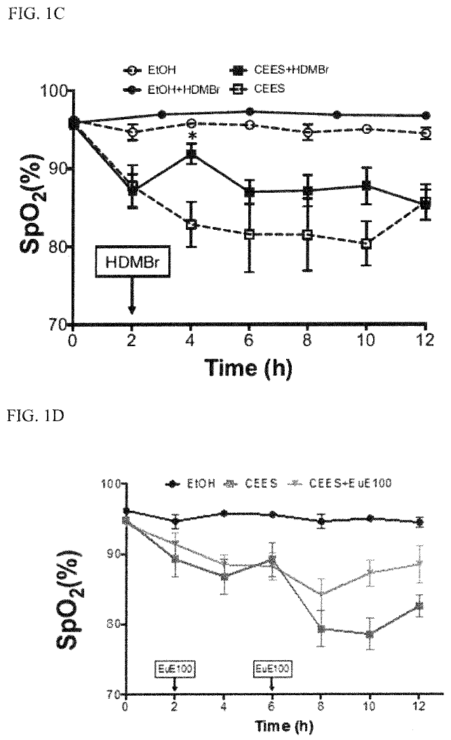

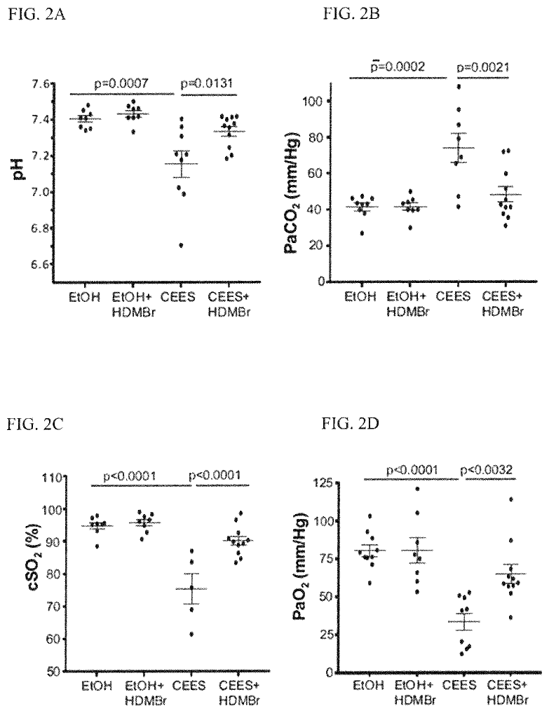

CEES exposure resulted in a significant decrease in arterial blood pH as compared to ethanol-exposed rats (7.15±0.07 vs 7.40±0.01) (FIG. 2A). CEES exposure increased PaCO₂ (73.96±8.03 mm/Hg) as compared to ethanol-exposed rats (FIGS. 2B and 3A). CEES exposure decreased cSO₂ (75.3%), PaO₂ (33.46±5.58 mm/Hg), and the PaO₂/FiO₂ ratio (150 in CEES-exposed animals, which is consistent with hypoxemia in ARDS as compared to ethanol-exposed rats.

Treatment with the NAS HDMBr (10 mg/kg, administered intraperitoneally 2 hours after CEES exposure) resulted in improvement in all ABG measurements in this animal model of ARDS (FIG. 2A-2E). HDMBr treatment reversed the drop in arterial blood pH (7.33±0.02; FIG. 2A). Similarly, the increased PaCO₂ following CEES exposure was significantly decreased by HDMBr treatment (47.29±3.97 mm/Hg; FIG. 2B). Improvement in ABG parameters following HDMBr treatment is further supported by calculated oxygen saturation (cSO₂) and partial pressure of arterial oxygen (PaO₂) assessments. While CEES exposure decreased cSO₂ and PaO₂ as described above, HDMBr treatment significantly improved cSO₂ (90.03%) and arterial PaO₂ (64.62±5.58 mm/Hg) levels (FIG. 2C-2D). Finally, treatment with HDMBr significantly improved the PaO₂/FiO₂ ratio to above 300 (FIG. 2E). A PaO₂/FiO₂ ratio of <300 is characteristic of ARDS.

Similar results were obtained after treatment with the NAS EuE100 (100 mg/kg, administered intraperitoneally 2 and 6 hours after CEES exposure). As seen above with the NAS HDMBr, the NAS EuE100 significantly reversed the increase in PaCO₂ caused by CEES exposure (FIG. 3A), significantly reversed the decrease in PaO₂, cSO₂, and PaO₂/FiO₂ ratio caused by CEES exposure (FIGS. 3B, 3C, and 3D, respectively). Similar improvements in blood gas values were observed in the gastric aspiration model after HDMBr treatment (data not shown).

### Example 3—Treatment with a Nucleic Acid Scavenger Decreases the Steady-State mRNA Levels of Pro-Inflammatory Genes and Increase the Steady-State mRNA Levels of Anti-Inflammatory Genes in the Lungs in the CEES Inhalation Model of Acute Respiratory Distress Syndrome

ARDS is also associated with increased systemic and pulmonary inflammation. To assess the role of inflammation in ARDS, the levels of pro-inflammatory cytokines and anti-inflammatory cytokines were examined. This example shows that while CEES exposure caused increased mRNA levels of inflammatory cytokines, treatment with NASs substantially reduced mRNA levels of these inflammatory cytokines in the lung. The same animal model system described in Example 1 was used in this experiment. At the end of study (12 h), realtime RT-PCR was carried out to determine steady-state mRNA levels of inflammation related genes in the lung tissue using Taqman primers and probes. Relative changes in mRNA levels for selected inflammatory cytokines were determined (normalized to β-actin mRNA). Data were analyzed by one-way ANOVA followed by Bonferroni multiple comparison test. Values represent mean±SEM; p-values for various group comparisons are provided.

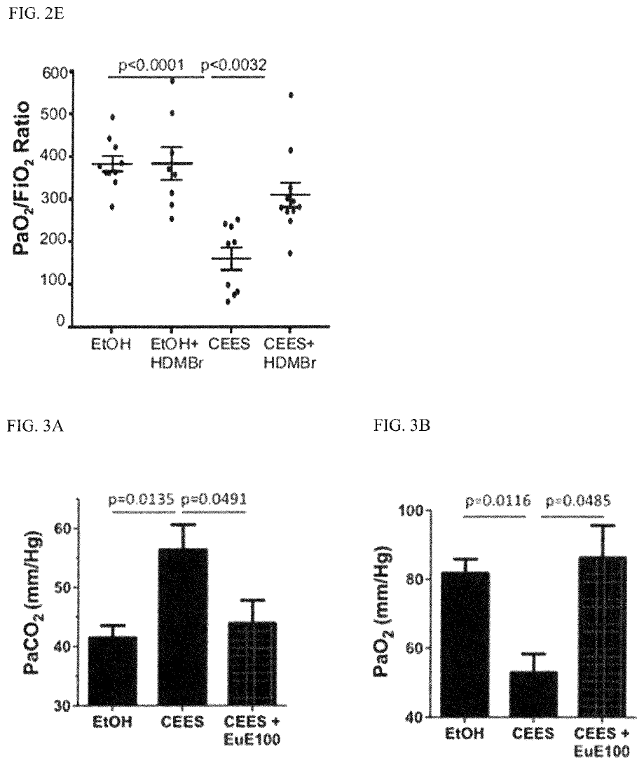

FIGS. 4A-D show that CEES treatment induced a significant increase in mRNA steady-state levels of the pro-inflammatory cytokines IL-6, IL1A, CXCL1, and CCL-2 (FIG. 4A-D). CEES treatment did not alter mRNA steady-state levels of the protective cytokine IL-4 (FIG. 4E).

Treatment with the NAS HDMBr (10 mg/kg, administered intraperitoneally 2 hours after CEES exposure) resulted in a significant decrease in mRNA steady-state levels of the pro-inflammatory cytokines IL-6, IL1A, CXCL1, and CCL-2 (FIG. 4A-4D) as compared to CEES-exposed animals. Interestingly, while CEES treatment did not alter mRNA steady-state levels of the protective cytokine IL-4, HDMBr treatment increased the mRNA steady-state levels of IL-4, indicating a role in ARDS (FIG. 4E).

The effect of the NAS EuE100 on the levels of pro-inflammatory cytokines was also investigated. Relative changes in mRNA levels for selected pro-inflammatory cytokines were determined (normalized to β-actin mRNA). Treatment with the NAS EuE100 (100 mg/kg, administered intraperitoneally 2 and 6 hours after CEES exposure) resulted in a significant decrease in mRNA steady-state levels of the pro-inflammatory cytokines IL-6 and CXCL1 (FIG. 4F-4G) as compared to CEES-exposed animals.

This example demonstrates that NAS treated animals showed improved inflammatory responses as demonstrated by decreased mRNA steady-state levels of the pro-inflammatory cytokines and increased mRNA steady-state levels of the anti-inflammatory cytokines. Therefore, NASs protect against enhanced inflammatory responses commonly observed in ARDS.

### Example 4—Treatment with a Nucleic Acid Scavenger Mitigates the Disruption of Alveolar-Capillary Barrier in the CEES Inhalation Model of Acute Respiratory Distress Syndrome

Another characteristic feature of ARDS is the disruption of the alveolar-capillary membrane, which among other effects causes protein to leak from the lung. As expected, inhaled CEES caused an increase in protein concentration in the bronchoalveolar lavage fluid (BALF). Vascular leak, bronchial cast formation and subsequent obstruction of the airways is a distinct injury pattern of CEES inhalation (10, 30, 31). The effect of NASs on the disruption of the alveolar-capillary membrane was determined in an in vivo model of ARDS. The same animal model system described in Example 1 was used in this experiment. At the end of study (12 h), BALF supernatants were analyzed for total protein concentration and IgM concentration to assess the alveolar-capillary barrier. Data were analyzed by one-way ANOVA followed by Bonferroni multiple comparison test. Values represent mean±SEM; p-values for various group comparisons are provided.

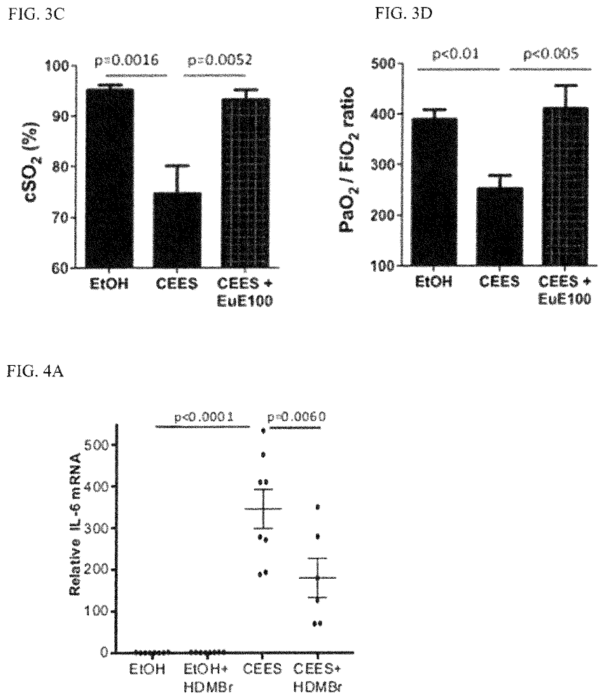

CEES exposure resulted in an increase in total protein concentration in BALF (FIGS. 5A and 5C) and IgM concentration in BALF (FIG. 5B). Treatment with the NAS HDMBr (10 mg/kg, administered intraperitoneally 2 hours after CEES exposure) resulted in a significant decrease in total protein concentration in BALF (FIG. 5A) and IgM concentration in BALF (1411±179 ng/ml for CEES group versus 930.0±29.30 ng/ml for the HDMBr treatment group; FIG. 5B) as compared to CEES-exposed animals.

Treatment with the NAS EuE100 (100 mg/kg, administered intraperitoneally 2 and 6 hours after CEES exposure) resulted in a significant decrease in total protein concentration in BALF (FIG. 5C) as compared to CEES-exposed animals.

This example demonstrates that NAS treated animals showed improved barrier function and mitigated leak of alveolar-capillary membrane as demonstrated by decreased protein concentration in BALF. Therefore, NASs protect against the disruption of alveolar-capillary barrier in ARDS.

### Example 5—Treatment with a Nucleic Acid Scavenger Decreases the Steady-State Levels of Coagulation Genes in the Lungs in the CEES Inhalation Model of Acute Respiratory Distress Syndrome

Increased clot formation resulting from activation of the coagulation pathway is a recognized marker of ARDS. Tissue factor (TF) is an important component of the coagulation pathway as TF initiates clot formation. TF expression can increase in the lung under certain pro-coagulant states (37, 38), such as ARDS. Other molecules, including plasminogen activator inhibitor (PAI-1) and thrombin-activatable fibrinolysis inhibitor (TAFI), inhibit degradation of blood clots by inhibiting fibrinolysis.

This example shows CEES exposure increased the mRNA levels of pro-coagulation proteins TF and PAI-1 and that treatment with NASs substantially reduced mRNA levels of these pro-coagulation proteins. The same animal model system described in Example 1 was used in this experiment. At the end of study (12 h), realtime RT-PCR was carried out to determine steady-state mRNA levels of coagulation related genes in the lung tissue using Taqman primers and probes. Relative changes in mRNA levels for TF and PAI-1—are shown after normalization with 18S mRNA (HDMBr) or and for TF after normalization with β-actin mRNA (EuE100). Data were analyzed by one-way ANOVA followed by Bonferroni multiple comparison test. Values represent mean±SEM; p-values for various group comparisons are provided.

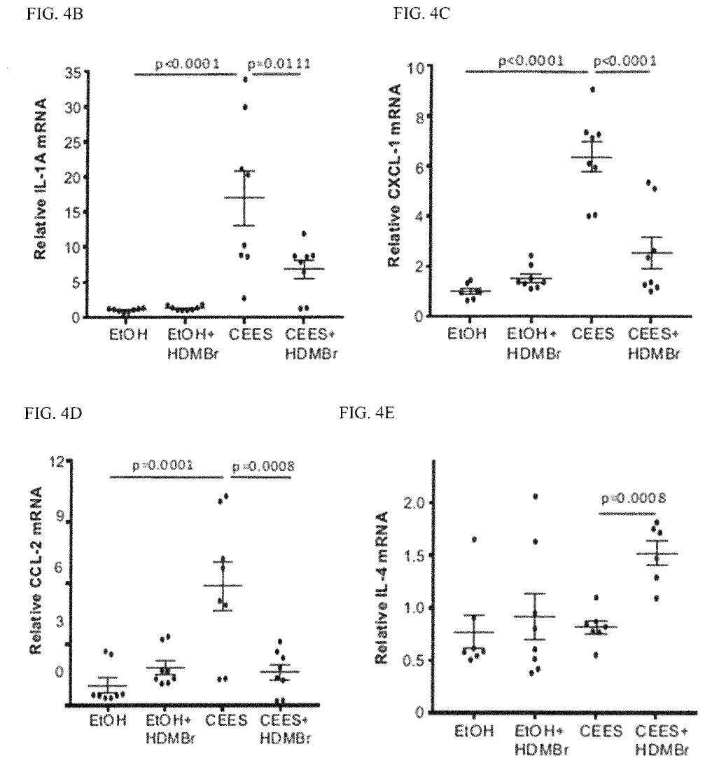

As shown in FIGS. 6A and 6B, CEES exposed rats had increased levels of tissue factor (TF) and PAI-1 mRNA 12 hours after exposure to CEES as compared to the ethanol treated control group. Treatment with the NAS HDMBr (10 mg/kg, administered intraperitoneally 2 hours after CEES exposure) resulted in a significant decrease in TF and PAI-1 mRNA levels as compared to CEES-exposed animals.

Treatment with the NAS EuE100 (100 mg/kg, administered intraperitoneally 2 and 6 hours after CEES exposure) also resulted in a significant decrease in TF mRNA levels as compared to CEES-exposed animals (FIG. 6C).

This example demonstrates that NAS treated animals showed reduced clot formation as demonstrated by decreased mRNA levels of the pro-coagulation factors. Therefore, NASs protect against the detrimental effects of clot formation as commonly observed in ARDS.

### Example 6—Treatment with a Nucleic Acid Scavenger Decreases Inflammatory Response in the CEES Inhalation Model of Acute Respiratory Distress Syndrome

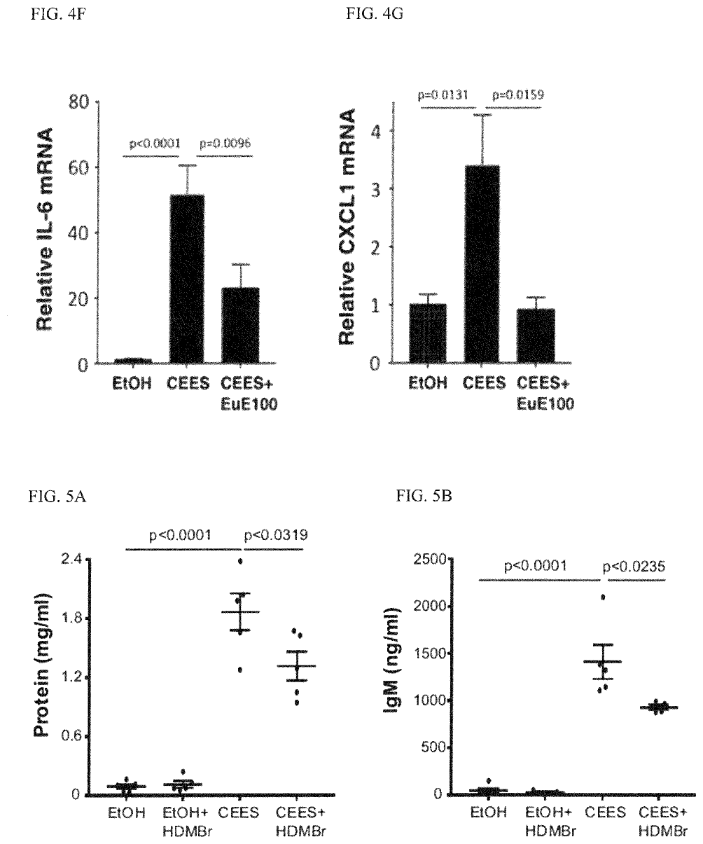

As discussed herein, ARDS is associated with increased systemic and pulmonary inflammation. The systemic and pulmonary inflammation observed in ARDS is induced after CEES exposure in the animal model described herein. To investigate the effects of NASs in ARDS, the inflammatory profile in BALF of animals exposed to CEES was examined by determining the expression of high mobility group box 1 protein (HMGB1) (FIG. 7A). Damage associated molecular patterns (DAMPs), such as HMGB1, are released in response to lung injury (as seen in ARDS) and serve as markers of inflammatory response (36).

The same animal model system described in Example 1 was used in this experiment. At the end of study (12 h), HMGB1 was quantified in BALF by western blot analysis to assess the degree of inflammatory response. This example shows CEES exposure generally increased the inflammatory response (as measured by HMGB1 levels) and that treatment with NASs reduced the inflammatory response induced by CEES exposure. Values represent mean±SEM.

The results are shown in FIG. 7. The top portion of FIG. 7 shows a representative western blot and the bottom portion shows densitometry analysis of western blots from two independent experiments. CEES exposed rats showed an increase in HMGB1 levels in BALF compared to the ethanol treated control group. Treatment with the NAS HDMBr (10 mg/kg, administered intraperitoneally 2 hours after CEES exposure) resulted in a significant decrease in HMGB1 protein levels as compared to CEES-exposed animals (FIG. 7).

This example demonstrates that NAS treated animals showed reduced inflammatory response and that NASs protect against the detrimental effects increased inflammatory response as commonly observed in ARDS.

### Example 7—Treatment with a Nucleic Acid Scavenger Decreases Fibrin Levels in BALF and Airway Obstruction in the CEES Inhalation Model of Acute Respiratory Distress Syndrome

ARDS is characterized by airway obstruction due to formation of fibrin rich casts. Such airway obstruction often leads to death. CEES exposed animals also display a hypercoaguable phenotype and mimic the airway obstruction found in ARDS. The effect of NASs on airway obstruction was examined in the animal model of ARDS using immunohistochemical staining of the lung for fibrin. Animals were scored on the percent of airways positive for fibrin casts.

This example shows CEES exposure generally increased the airway obstruction and that treatment with NASs reduced airway obstruction induced by CEES exposure. The same animal model system described in Example 1 was used in this experiment. At the end of study (12 h), rats were euthanized and inflated lungs fixed with 4% paraformaldehyde in PBS. Airway obstruction was assessed by staining sections of right middle and right lower lobes with antibodies against fibrin. Immunohistochemical staining of fibrin shown in brown. Upper panels: 5× magnification; Lower panel: 20× magnification.

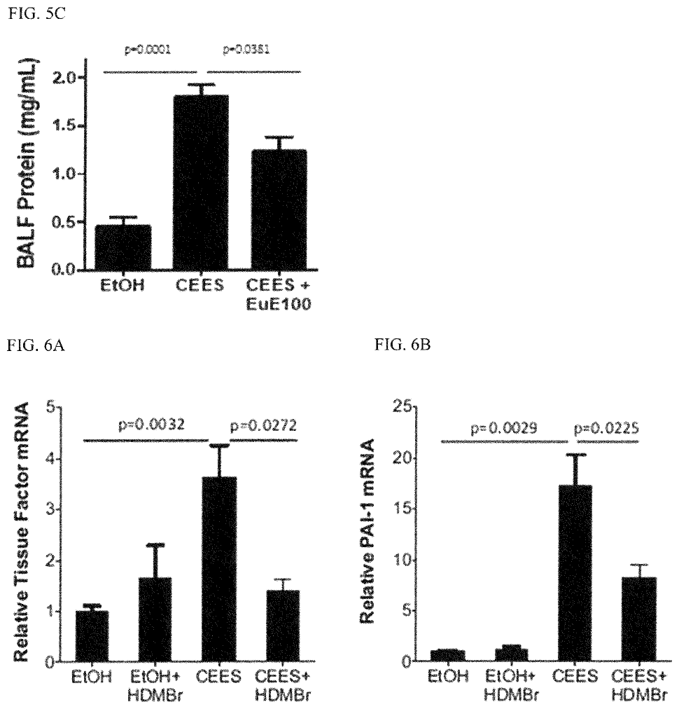

Serial lung sections of H&E and fibrin stained slides showed intense staining of casts for fibrin within the airway of the CEES-exposed animals (FIG. 8A). There was also evidence of edema and significant peribronchial and perivascular staining for fibrin. Airways were scored for fibrin casts by counting for the presence or absence of positive staining at 5× magnification. A minimum of five fields per section were counted. Percent of airways positive for fibrin casts were plotted (FIG. 8B). Quantification showed that more than 50% of the airways stained positive for fibrin casts. In the HDMBr treatment group (10 mg/kg, administered intraperitoneally 2 hours after CEES exposure) there was absence of casts in a significant number of airways analyzed. In addition, the extent of cast formation within an airway was reduced with HDMBr treatment (FIGS. 8A and 8B). However, there was still some perivascular edema that also contained fibrin. In addition, although there was minimal peribronchial edema, fibrin staining was still significant. Values represent mean±SEM; * indicates p<0.05 from the untreated CEES-exposed animals.

To further quantify levels of fibrin in the airways, western blot analysis for fibrin in the BALF supernatant was conducted and quantified using densitometry. There was marked decrease in fibrin band intensity in the HDMBr treated group when compared to the CEES-exposed group and the control groups (FIG. 8C). Values represent mean±SEM (n=5-6 per group); p-values for various group comparisons are provided.

This example demonstrates that NAS treated animals showed reduced airway obstruction as demonstrated by decreased levels of fibrin cases and decreased fibrin protein levels in BALF. Therefore, NASs protect against the detrimental effects of airway obstruction as commonly observed in ARDS.

### Example 8—Treatment with a Nucleic Acid Scavenger Decreased Levels of Circulating Nucleic Acids in the CEES Inhalation Model of Acute Respiratory Distress Syndrome

ARDS is characterized by increased inflammation, vascular leak and enhanced coagulation in airways leading to acute airway obstruction, respiratory failure and death. These same symptoms are observed in rats exposed to CEES (10, 30, 31). The presence of circulating nucleic acids has been observed concurrently with the activation of the inflammatory and coagulation pathways. The effects of NASs in an animal model of ARDS was examined by determining the levels of circulating nucleic acids in BALF and platelet free plasma samples from rats exposed to CEES.

This example shows CEES exposure generally increased the levels of circulating nucleic acids and that treatment with NASs reduced the levels of circulating nucleic acids induced by CEES exposure. The same animal model system described in Example 1 was used in this experiment. At the end of study (12 h), rats were euthanized and BALF and plasma (platelet free) samples were collected. Total circulating nucleic acids were isolated as described in the Methods section. Data were analyzed by one-way ANOVA followed by Bonferroni multiple comparison test. Values represent mean±SEM (n=5-6 per group); p-values for various group comparisons are provided.

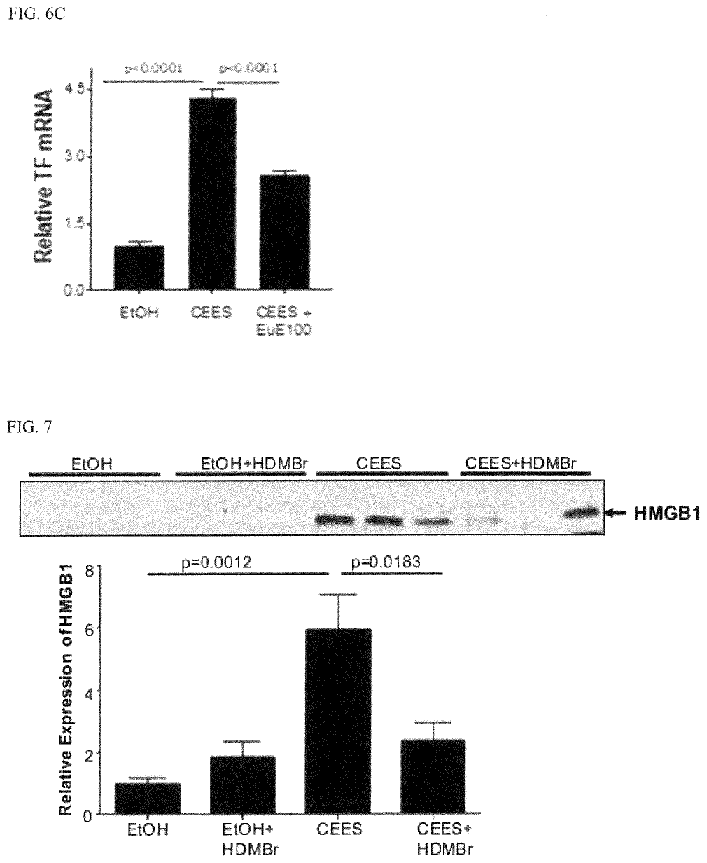

CEES exposure caused an increase in circulating levels of extracellular nucleic acids both in the BALF and plasma of rats (FIGS. 9A and 9B, respectively). In order to understand the role of increased levels of these nucleic acids and whether they contribute to the pathogenesis or ARDS, the effect of NASs was examined. Administration of the NAS HDMBr (10 mg/kg, administered intraperitoneally 2 hours after CEES exposure) decreased levels of total nucleic acids as assessed at 12 h post CEES exposure.

To evaluate mortality in this model system, animals were continuously monitored over a 12 h period and survival assessed using a Kaplan Meier curve and groups compared using the Mantel-Cox model. Data were analyzed by one-way ANOVA followed by Bonferroni multiple comparison test. In this animal model system, CEES exposure resulted in a 50% mortality within 12 hours of CEES administration (FIG. 9C). The administration of the NAS HDMBr as above completely prevented mortality at this time point (FIG. 9C).

This example demonstrates that NAS treated animals showed reduced levels of circulating nucleic acids in an animal model of ARDS. Importantly, NAS treated animals showed a 100% survival rate (compared to a survival rate of approximately 50% in CEES treated animals) at the 12 hour time point.

### Example 9—Extracellular Nucleic Acid from CEES-Exposed Rats Increased Inflammatory Cytokines in Lung Epithelial Cells

Nucleic acids can interact with cell surface receptors and activate a diverse range of signaling pathways. In order to explore the role of extracellular nucleic acid in ARDS, total extracellular nucleic acid from BALF of CEES-exposed rats was isolated. Total nucleic acid was isolated from the BALF supernatant of CEES-exposed animals (12 hours after exposure), pooled, and added exogenously at 1.5 and 2.5 μg/ml to cultured human airway epithelial cells in vitro. The CEES exposed animals were treated as described in Example 1. After 6 hours total RNA was isolated from cells and the mRNA levels of the pro-inflammatory cytokine IL-6 and CXCL1 were determined by realtime RT-PCR using specific Taqman primers and probes. Data were analyzed by one-way ANOVA followed by Bonferroni multiple comparison test. Values represent mean±SEM.

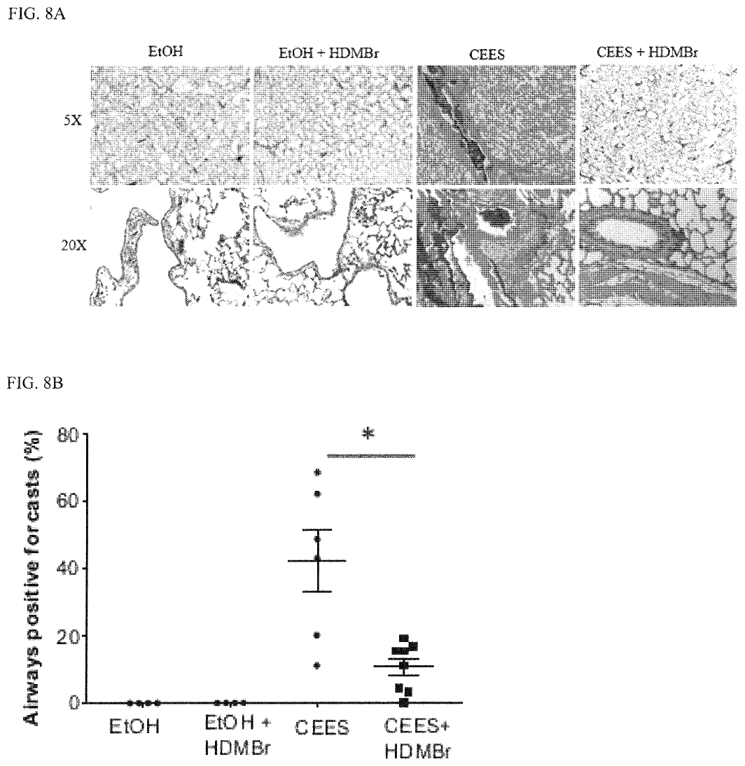

As shown in FIGS. 10A and 10B, addition of extracellular nucleic acid isolated from the BALF of CEES-exposed animals dose dependently increased mRNA steady state levels of IL-6 and CXCL1, respectively.

The effect of the NAS HDMBr on the increase in CXCL1 mRNA levels induced by extracellular nucleic acid in BALF from CEES treated animals was determined. Total nucleic acid was isolated from the BALF supernatant of CEES-exposed animals (12 hours after exposure) and pooled. 2.5 μg/ml isolated nucleic acid in the presence or absence of HDMBr (10 μg/ml) was added to cultured human airway epithelial cells. After 6 hours total RNA was isolated from cells and analyzed for mRNA levels by realtime RT-PCR using specific Taqman primers and probes. Data were analyzed by one-way ANOVA followed by Bonferroni multiple comparison test. Values represent mean±SEM.

Incubation of the isolated extracellular nucleic acids from BALF of CEES treated animals (2.5 μg/ml) with HDMBr (10 μg/ml) resulted in decreased levels of CXCL1 as compared to CEES exposed animals (FIG. 10C).

This example demonstrates that extracellular nucleic acids generated during ARDS are active in the inflammatory cascade and that NASs are capable of reducing this effect of extracellular nucleic acids.

### Example 10—Treatment with a Nucleic Acid Scavenger Diminished Lung Injury and Prevented Mortality in the CEES Inhalation Model of Acute Respiratory Distress Syndrome

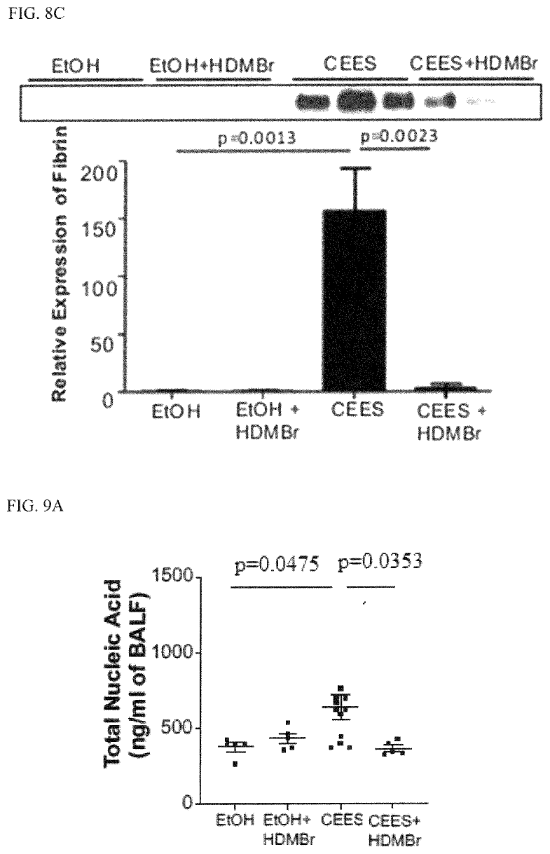

ARDS is characterized by acute lung injury. CEES exposure mimics this acute lung injury. CEES exposed rats demonstrated epithelial sloughing, perivascular and peribronchial fluid accumulation, apoptosis, extravasation of RBCs, bronchial cast formation, accumulation of proteinaceous debris in the airspaces, alveolar septal thickening and neutrophil accumulation in the alveolar and interstitial spaces (FIG. 11).

The animal model used is that described in Example 1. At the end of study (12 h), rats were euthanized and inflated lungs fixed with 4% paraformaldehyde in PBS. Lungs were sectioned and stained with hematoxylin and eosin dyes. Images were acquired using a Leica brightfield microscopy. A representative section from 3-4 rats per treatment group is shown in FIG. 11 (black arrow indicates airway casts; black arrowheads show epithelial sloughing; green arrow indicates edema and inflammatory cell infiltrations. Upper panels: 5× magnification; Lower panel: 20× magnification).

A single treatment with HDMBr showed improvement in acute lung injury in this animal model of ARDS. Acute mortality assessed at 12 h was completely prevented in CEES-exposed rats that were treated with HDMBr (FIG. 9C). This complete prevention of mortality was accompanied by a decrease in total extracellular nucleic acids in the HDMBr treated group (as compared to the CEES treated group). Taken together, these findings demonstrate efficacy of NASs in mitigating lung injury and other pathology associated with ARDS.

### Example 11—Extracellular Nucleic Acid is Increased in the BALF of Rats Exposed to Gastric Aspirate and HDMBr Reduces these Levels

To determine if extracellular nucleic levels are increased in an additional animal model of ARDS, the gastric aspiration rat model (as described in the Methods section) was used. Animals were exposed to GA as described, followed by HDMBr or saline administration (2 hours post GA exposure). Animals were sacrificed 6 h post exposure. To determine extracellular nucleic acid levels, total nucleic acid was isolated from 200 μl of BALF. Extracellular nucleic acid in the BALF was measured with a spectrophotometer as described in the Methods section.

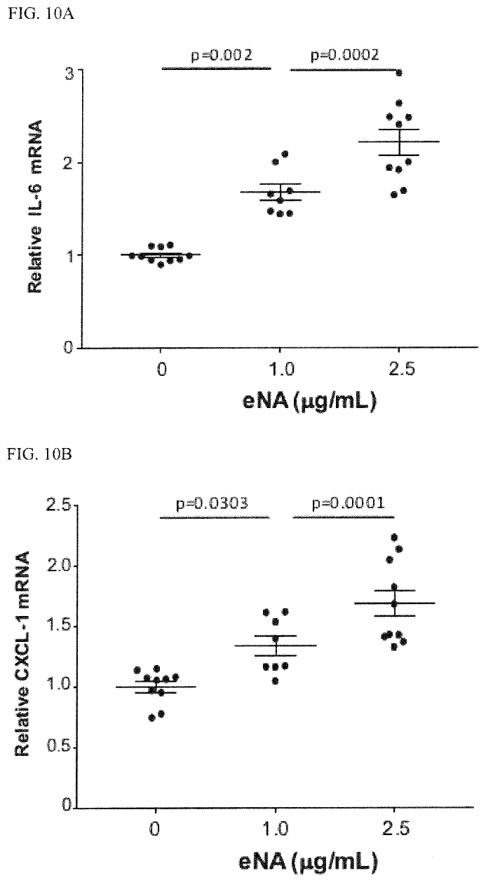

The results are shown in FIG. 13. Administration of GA significantly increased extracellular nucleic acid levels as compared to control animals (no GA administration). The increase in extracellular nucleic acid after GA administration indicates this system is a useful model for studying ARDS. In addition, FIG. 13 shows administration of 10 mg/kg HDMBr inhibited the GA-induced increased in extracellular nucleic acid. Data were analyzed by one-way ANOVA followed by Bonferroni multiple comparison test. Values are presented as mean±SEM (n≥6 in each group), * indicates significant (p<0.05) difference from GA exposed rats.

This example demonstrates that NAS treated animals showed decreased levels of extracellular nucleic acid. As shown in Example 8, increased levels of extracellular nucleic acid are associated with increased mortality in the CEES aerosol exposure model.

### Example 12—HDMBr Administration Reduces Inflammation in the Gastric Aspiration Model of ARDS

ARDS is associated with increased systemic and pulmonary inflammation. To assess the role of inflammation in ARDS in the GA model, the levels of pro-inflammatory cytokines and anti-inflammatory cytokines were examined. This example shows that GA exposure caused increased mRNA levels of inflammatory cytokines and treatment with NASs substantially reduced mRNA levels of these inflammatory cytokines in the lung.

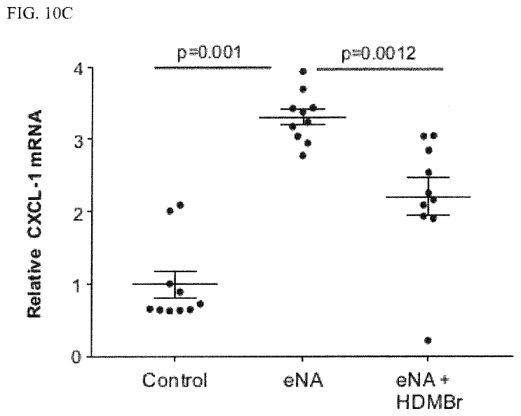

The animal model used is the GA model described in the Methods section. Animals were exposed to GA as described, followed by HDMBr (10 mg/kg) or saline administration 2 hours post GA exposure. Animals were sacrificed 6 h post exposure and lung tissue was collected. Total RNA was isolated from the lung and realtime RT PCR was performed to assess the levels of the pro-inflammatory genes IL-6 (FIG. 14A), IL1A (FIG. 14B), CXCL-1 (FIG. 14C) and CCL-2 (FIG. 14D). Relative changes in mRNA levels for the pro-inflammatory cytokine genes are shown after normalization with β-actin mRNA.

The results are shown in FIGS. 14A-D. The data show that GA administration dramatically increased the levels of IL-6, IL1A, CXCL-1, and CCL-2 mRNA in the lungs consistent with induction of inflammation in this model. HDMBr (10 mg/kg) significantly reduced GA-stimulated increases in inflammatory cytokine mRNA levels. Data were analyzed by one-way ANOVA followed by Bonferroni multiple comparison test. Values are presented as mean±SEM (n=5-6 in each group).

This example demonstrates that NAS treated animals showed improved inflammatory responses as demonstrated by decreased steady-state mRNA levels of the pro-inflammatory cytokines. Therefore, NASs protect against enhanced inflammatory responses commonly observed in ARDS.

### Example 13—Treatment with a Nucleic Acid Scavenger Mitigates the Disruption of Alveolar-Capillary Barrier in the Gastric Aspiration Model of ARDS

As discussed in Example 4, a characteristic feature of ARDS is the disruption of the alveolar-capillary membrane, which among other effects causes protein to leak from the lung. The effect of NASs on the disruption of the alveolar-capillary membrane in the GA aspiration model was determined.

The animal model used is the GA model described in the Methods section. Animals were exposed to GA as described, followed by HDMBr (10 mg/kg) or saline administration 2 hours post GA exposure. Animals were sacrificed 6 h post exposure and BALF supernatants were analyzed for total protein and IgM concentration to assess the alveolar-capillary barrier and vascular leak.

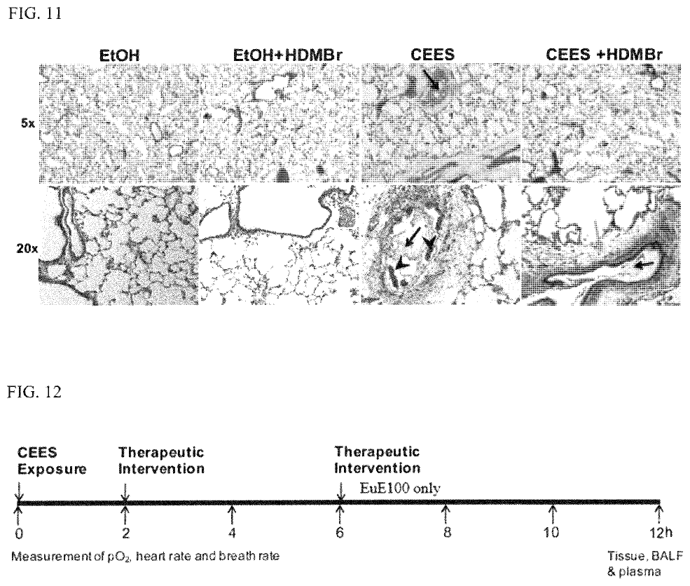

The results are shown in FIGS. 15A-B. The data show that GA administration resulted in an increase in total protein concentration in BALF (FIG. 15A) and IgM concentration in BALF (FIG. 15B). Treatment with the NAS HDMBr resulted in a significant decrease in total protein concentration in BALF (FIG. 15A) and IgM concentration in BALF (FIG. 15B) as compared to GA-exposed animals. Data were analyzed by one-way ANOVA followed by Bonferroni multiple comparison test. Values represent mean±SEM (n=5-6 in each group); * indicates significant (p<0.05) difference from GA exposed rats.

This example demonstrates that NAS treated animals showed improved barrier function as demonstrated by decreased protein concentration (both total protein and IgM) in BALF. Therefore, NASs protect against the disruption of alveolar-capillary barrier in ARDS.

### Example 14—Treatment with a Nucleic Acid Scavenger Decreases Inflammatory Response in the Gastric Aspiration Model of ARDS

ARDS is associated with increased systemic and pulmonary inflammation. The systemic and pulmonary inflammation observed in ARDS is induced after GA administration. To investigate the effects of NASs in ARDS, the inflammatory profile in BALF of animals exposed to GA was examined using expression of high mobility group box 1 protein (HMGB1). Damage associated molecular patterns (DAMPs), such as HMGB1, are released in response to lung injury (as seen in ARDS) and serve as markers of inflammatory response (36).

This example shows GA exposure generally increased the inflammatory response in the lung and treatment with NASs reduced the inflammatory response induced by GA exposure. The animal model used is the GA model described in the Methods section. Animals were exposed to GA as described, followed by HDMBr (10 mg/kg) or saline administration 2 hours post GA exposure. Animals were sacrificed 6 h post exposure and BALF supernatant was collected to assess HMGB1 levels. HMGB1 was quantified in BALF by western blot analysis to assess the degree of inflammatory response.

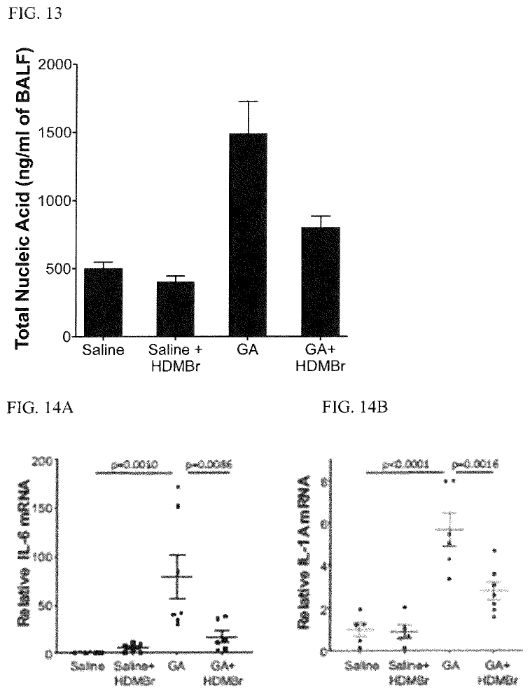

The results are shown in FIG. 16. GA exposed rats showed an increase in HMGB1 levels in BALF compared to the saline treated control group. Treatment with the NAS HDMBr resulted in a significant decrease in HMGB1 protein levels as compared to GA-exposed animals (FIG. 16).

This example demonstrates that NAS treated animals showed reduced inflammatory response and that NASs protect against the detrimental effects increased inflammatory response as commonly observed in ARDS.

### Example 15—Treatment with a Nucleic Acid Scavenger Mitigates the Disruption of Alveolar-Capillary Barrier in the Gastric Aspiration Model of ARDS

As shown in Example 4, HDMBr and EuE100 improved barrier function and mitigated leak of alveolar-capillary membrane as demonstrated by decreased protein concentration in BALF in the CEES animal model. The effect of NASs on the disruption of the alveolar-capillary membrane was determined in the gastric aspiration model of ARDS. The animal model used is the GA model described in the Methods section. Animals were exposed to GA as described, followed by HDMBr (10 mg/kg) or saline administration 2 hours post GA exposure. Animals were sacrificed 6 h post exposure and BALF supernatant was collected and analyzed for total protein concentration and IgM concentration to assess the alveolar-capillary barrier. Data were analyzed by one-way ANOVA followed by Bonferroni multiple comparison test. Values represent mean±SEM; p-values for various group comparisons are provided.

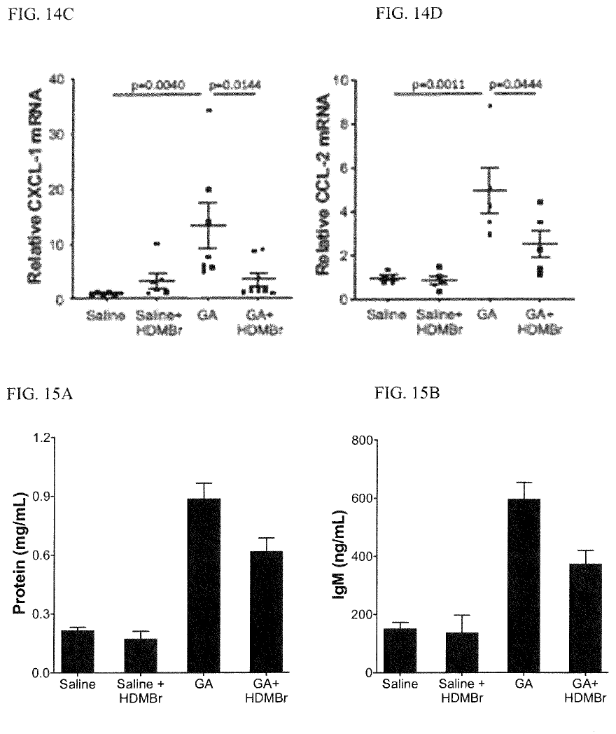

GA exposure resulted in an increase in total protein concentration in BALF (FIG. 17A) and IgM concentration in BALF (FIG. 17B). Treatment with the NAS HDMBr (10 mg/kg, administered intraperitoneally 2 hours after CEES exposure) resulted in a significant decrease in total protein concentration in BALF (FIG. 17A) and IgM concentration in BALF (FIG. 17B) as compared to CEES-exposed animals.

This example confirms the results of Example 4 is a different animal model of ARDS and demonstrates that NAS treated animals showed improved barrier function and mitigated leak of alveolar-capillary membrane as demonstrated by decreased protein concentration in BALF. Therefore, NASs protect against the disruption of alveolar-capillary barrier in ARDS.

### Example 16—Treatment with a Nucleic Acid Scavenger Decreases the Steady-State Levels of Coagulation Genes in the Lungs in the CEES Inhalation Model of Acute Respiratory Distress Syndrome

As discussed in Example 5, increased clot formation resulting from activation of the coagulation pathway is a recognized marker of ARDS and involves the activity of both TF and PAI-1. This example shows GA exposure increased the mRNA levels of pro-coagulation proteins TF and PAI-1 and that treatment with NASs substantially reduced mRNA levels of these pro-coagulation proteins.

The animal model used is the GA model described in the Methods section. Animals were exposed to GA as described, followed by HDMBr (10 mg/kg) or saline administration 2 hours post GA exposure. Animals were sacrificed 6 h post exposure and realtime RT-PCR was carried out to determine steady-state mRNA levels of coagulation related genes in the lung tissue using Taqman primers and probes. Relative changes in mRNA levels for TF and PAI-1 are shown after normalization with 18S mRNA. Data were analyzed by one-way ANOVA followed by Bonferroni multiple comparison test. Values represent mean±SEM; p-values for various group comparisons are provided.

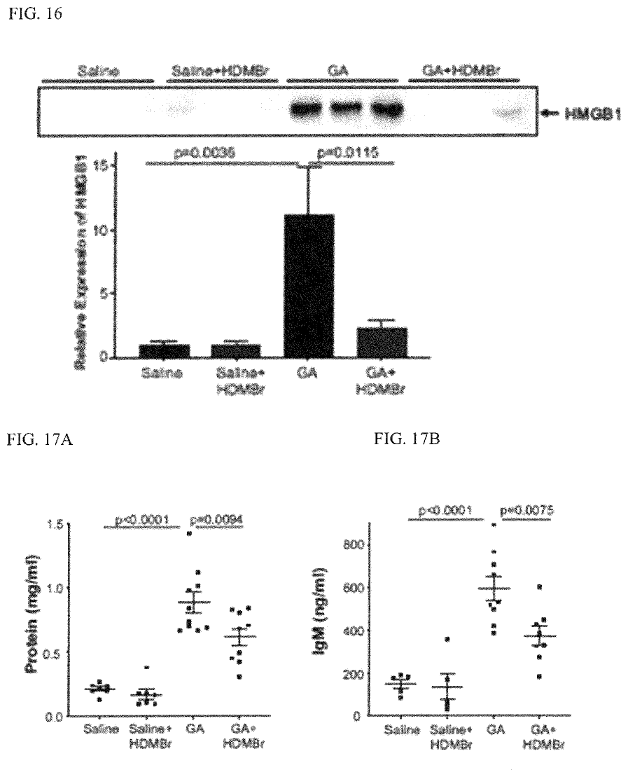

As shown in FIGS. 18A and 18B, GA exposed rats had increased levels of TF and PAI-1 mRNA 6 hours after exposure to GA as compared to the saline treated control group. Treatment with the NAS HDMBr (10 mg/kg, administered intraperitoneally 2 hours after GA exposure) resulted in a significant decrease in TF and PAI-1 mRNA levels as compared to GA-exposed animals.

This example demonstrates that NAS treated animals showed reduced clot formation as demonstrated by decreased mRNA levels of the pro-coagulation factors TF and PAI-1. Therefore, NASs protect against the detrimental effects of clot formation as commonly observed in ARDS.

### Example 17—Treatment with a Nucleic Acid Scavenger Decreases Fibrin Levels in BALF in the Gastric Aspiration Model of Acute Respiratory Distress Syndrome

As discussed in Example 7, ARDS is characterized by airway obstruction due to formation of fibrin rich casts. Such airway obstruction often leads to death. GA exposed animals also display a hypercoaguable phenotype and mimic the airway obstruction found in ARDS. The effect of NASs on airway obstruction was examined in the GA model of ARDS using immunohistochemical staining of the lung for fibrin. Animals were scored on the percent of airways positive for fibrin casts.

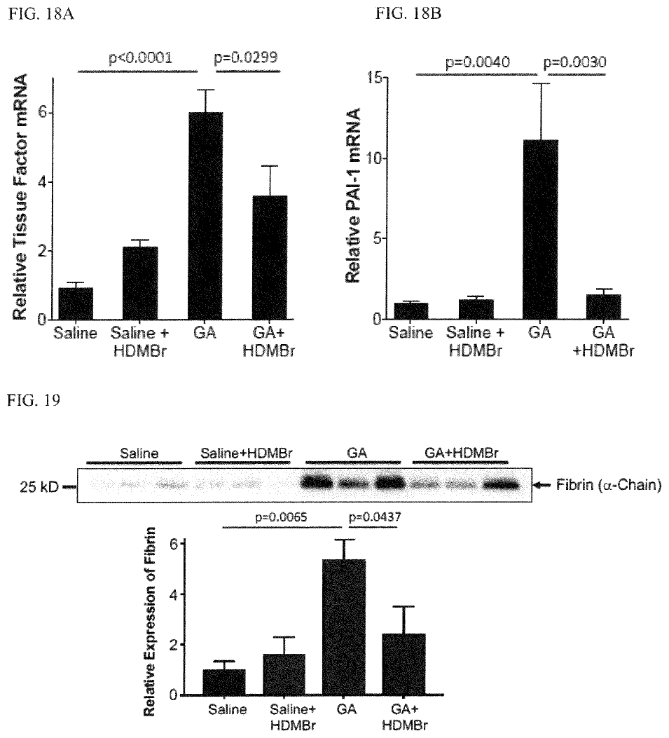

The animal model used is the GA model described in the Methods section. Animals were exposed to GA as described, followed by HDMBr (10 mg/kg) or saline administration 2 hours post GA exposure. Animals were sacrificed 6 h post exposure and BALF supernatant collected. Values represent mean±SEM (n=5-6 per group); p-values for various group comparisons are provided. To quantify levels of fibrin in the airways, western blot analysis for fibrin in the BALF supernatant was conducted and quantified using densitometry. Values represent mean±SEM; * indicates p<0.05 from the untreated GA-exposed animals. There was marked decrease in fibrin band intensity in the HDMBr treated group when compared to the GA-exposed group and the control groups (FIG. 19).

This example demonstrates that NAS treated animals showed reduced airway obstruction as demonstrated by decreased fibrin protein levels in BALF. Therefore, NASs protect against the detrimental effects of airway obstruction as commonly observed in ARDS.

### Example 18—Extracellular Nucleic Acid is Increased in the BALF of ARDS Patients

Total nucleic acid was isolated from 200 μl of archived BALF supernatant obtained from ARDS patients and from control samples subjects without ARDS. The patient samples were obtained from the ALTA trial (NCT00434993) through the NIH BioLINCC network. The control samples were obtained from the SPIROMICS study (Couper, et al., Thorax 2014; 69: 491-494) through the NIH BioLINCC network. Extracellular nucleic acid in the BALF was measured with a spectrophotometer as described in the Methods section. Values are presented as mean±SEM (n=15 in each group); * indicates significant (p<0.05) difference from control group.

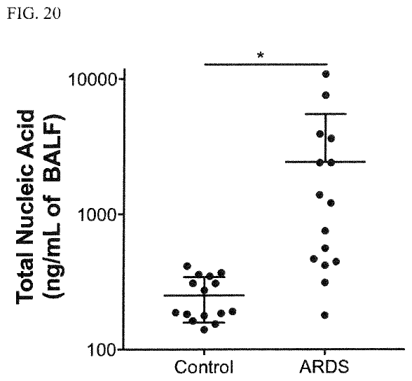

The results are shown in FIG. 20. The data show that in patients with ARDS, extracellular nucleic acid in BALF was significantly increased as compared to the control group. The values for the ARDS patient samples were 2439±792.7 versus 251.2±23.97 for controls. This example demonstrates that extracellular nucleic acid was increased in human subjects with ARDS.

Consistent with these results, extracellular nucleic acid was also shown to be increased in two distinct animal models of ARDS (see Examples 8 and 12).

### REFERENCES

1. Matthay M A, Ware L B, and Zimmerman G A. The acute respiratory distress syndrome. J Clin Invest. 2012; 122(8):2731-40.
2. Spieth P M, and Zhang H. Pharmacological therapies for acute respiratory distress syndrome. Curr Opin Crit Care. 2014; 20(1):113-21.
3. Aeffner F, Bolon B, and Davis I C. Mouse Models of Acute Respiratory Distress Syndrome: A Review of Analytical Approaches, Pathologic Features, and Common Measurements. Toxicol Pathol. 2015; 43(8):1074-92.
4. Force A D T, Ranieri V M, Rubenfeld G D, Thompson B T, Ferguson N D, Caldwell E, et al. Acute respiratory distress syndrome: the Berlin Definition. JAMA. 2012; 307(23):2526-33.
5. Ahmad S, and Ahmad A. Emerging targets for treating sulfur mustard-induced injuries. Ann NY Acad Sci. 2016; 1374(1):123-31.
6. McClintock S D, Hoesel L M, Das S K, Till G O, Neff T, Kunkel R G, et al. Attenuation of half sulfur mustard gas-induced acute lung injury in rats. J Appl Toxicol. 2006; 26(2): 126-31.
7. Pant S C, and Vijayaraghavan R. Histomorphological and histochemical alterations following short-term inhalation exposure to sulfur mustard on visceral organs of mice. Biomed Environ Sci. 1999; 12(3):201-13.
8. Calvet J H, Jarreau P H, Levame M, D'Ortho M P, Lorino H, Harf A, et al. Acute and chronic respiratory effects of sulfur mustard intoxication in guinea pig. J Appl Physiol (1985). 1994; 76(2):681-8.
9. Chevillard M, Lainee P, Robineau P, and Puchelle E. Toxic effects of sulfur mustard on respiratory epithelial cells in culture. Cell Biol Toxicol. 1992; 8(2):171-81.
10. Veress L A, O'Neill H C, Hendry-Hofer T B, Loader J E, Rancourt R C, and White C W. Airway obstruction due to bronchial vascular injury after sulfur mustard analog inhalation. American journal of respiratory and critical care medicine. 2010; 182(11): 1352-61.
11. Eisenmenger W, Drasch G, von Clarmann M, Kretschmer E, and Roider G. Clinical and morphological findings on mustard gas [bis(2-chloroethyl)sulfide] poisoning. J Forensic Sci. 1991; 36(6):1688-98.
12. Balali-Mood M, and Hefazi M. Comparison of early and late toxic effects of sulfur mustard in Iranian veterans. Basic Clin Pharmacol Toxicol. 2006; 99(4):273-82.
13. Kehe K, and Szinicz L. Medical aspects of sulphur mustard poisoning. Toxicology. 2005; 214(3): 198-209.
14. Kehe K, Thiermann H, Balszuweit F, Eyer F, Steinritz D, and Zilker T. Acute effects of sulfur mustard injury-Munich experiences. Toxicology. 2009; 263(1):3-8.
15. Sinclair D C. The clinical features of mustard-gas poisoning in man. Br Med J. 1948; 2(4570):290-4.
16. Schultz M J, Haitsma J J, Zhang H, and Slutsky A S. Pulmonary coagulopathy as a new target in therapeutic studies of acute lung injury or pneumonia—a review. Crit Care Med. 2006; 34(3):871-7.
17. Welty-Wolf K E, Carraway M S, Ortel T L, Ghio A J, Idell S, Egan J, et al. Blockade of tissue factor-factor X binding attenuates sepsis-induced respiratory and renal failure. Am J Physiol Lung Cell Mol Physiol. 2006; 290(1):L21-31.
18. Standiford T J, and Ward P A. Therapeutic targeting of acute lung injury and acute respiratory distress syndrome. Transl Res. 2016; 167(1):183-91.
19. Kannemeier C, Shibamiya A, Nakazawa F, Trusheim H, Ruppert C, Markart P, et al. Extracellular RNA constitutes a natural procoagulant cofactor in blood coagulation. Proc Natl Acad Sci USA. 2007; 104(15):6388-93.
20. Wygrecka M, Morty R E, Markart P, Kanse S M, Andreasen P A, Wind T, et al. Plasminogen activator inhibitor-1 is an inhibitor of factor VII-activating protease in patients with acute respiratory distress syndrome. J Biol Chem. 2007; 282(30):21671-82.
21. Bliksoen M, Mariero L H, Torp M K, Baysa A, Ytrehus K, Haugen F, et al. Extracellular mtDNA activates NF-kappaB via toll-like receptor 9 and induces cell death in cardiomyocytes. Basic Res Cardiol. 2016; 11(4):42.
22. Cunha C, Gomes C, Vaz A R, and Brites D. Exploring New Inflammatory Biomarkers and Pathways during LPS-Induced M1 Polarization. Mediators Inflamm. 2016; 2016:6986175.
23. Jimenez-Alcazar M, Kim N, and Fuchs T A. Circulating Extracellular DNA: Cause or Consequence of Thrombosis? Semin Thromb Hemost. 2017.
24. Pinti M, Cevenini E, Nasi M, De Biasi S, Salvioli S, Monti D, et al. Circulating mitochondrial DNA increases with age and is a familiar trait: Implications for “inflamm-aging”. Eur J Immunol. 2014; 44(5):1552-62.
25. Preissner K T, and Herwald H. Extracellular nucleic acids in immunity and cardiovascular responses: between alert and disease. Thromb Haemost. 2017; 117(7):1272-82.
26. Deindl E, Fischer S, and Preissner K T. New directions in inflammation and immunity: the multi-functional role of the extracellular RNA/RNase system. Indian J Biochem Biophys. 2009; 46(6):461-6.
27. Preissner K T. Extracellular RNA. A new player in blood coagulation and vascular permeability. Hamostaseologie. 2007; 27(5):373-7.
28. O'Driscoll L. Extracellular nucleic acids and their potential as diagnostic, prognostic and predictive biomarkers. Anticancer Res. 2007; 27(3A): 1257-65.
29. Pisetsky D S, Lee J, Leong K W, and Sullenger B A. Nucleic acid-binding polymers as anti-inflammatory agents: reducing the danger of nuclear attack. Expert Rev Clin Immunol. 2012; 8(1):1-3.
30. Rancourt R C, Ahmad A, Veress L A, Rioux J S, Garlick R B, and White C W. Antifibrinolytic mechanisms in acute airway injury after sulfur mustard analog inhalation. Am J Respir Cell Mol Biol. 2014; 51(4):559-67.
31. Rancourt R C, Veress L A, Ahmad A, Hendry-Hofer T B, Rioux J S, Garlick R B, et al. Tissue factor pathway inhibitor prevents airway obstruction, respiratory failure and death due to sulfur mustard analog inhalation. Toxicology and applied pharmacology. 2013; 272(1):86-95.
32. Lee J, Sohn J W, Zhang Y, Leong K W, Pisetsky D, and Sullenger B A. Nucleic acid-binding polymers as anti-inflammatory agents. Proc Natl Acad Sci USA. 2011; 108(34):14055-60.
33. Veress L A, Hendry-Hofer T B, Loader J E, Rioux J S, Garlick R B, and White C W. Tissue plasminogen activator prevents mortality from sulfur mustard analog-induced airway obstruction. Am J Respir Cell Mol Biol. 2013; 48(4):439-47.
34. Rice T W, Wheeler A P, Bernard G R, Hayden D L, Schoenfeld D A, Ware L B, et al. Comparison of the SpO2/FIO2 ratio and the PaO2/FIO2 ratio in patients with acute lung injury or ARDS. Chest. 2007; 132(2):410-7.
35. Matute-Bello G, Downey G, Moore B B, Groshong S D, Matthay M A, Slutsky A S, et al. An official American Thoracic Society workshop report: features and measurements of experimental acute lung injury in animals. Am J Respir Cell Mol Biol. 2011; 44(5):725-38.
36. Yang H, Wang H, Czura C J, and Tracey K J. HMGB1 as a cytokine and therapeutic target. J Endotoxin Res. 2002; 8(6):469-72.
37. Idell S. Coagulation, fibrinolysis, and fibrin deposition in acute lung injury. Crit Care Med. 2003; 31(4 Suppl):S213-20.
38. Mackman N, Sawdey M S, Keeton M R, and Loskutoff D J. Murine tissue factor gene expression in vivo. Tissue and cell specificity and regulation by lipopolysaccharide. Am J Pathol. 1993; 143(1):76-84.
39. Jain S, Pitoc G A, Holl E K, Zhang Y, Borst L, Leong K W, et al. Nucleic acid scavengers inhibit thrombosis without increasing bleeding. Proc Natl Acad Sci USA. 2012; 109(32):12938-43.
40. Komissarov A A, Florova G, and Idell S. Effects of extracellular DNA on plasminogen activation and fibrinolysis. J Biol Chem. 2011; 286(49):41949-62.
41. Barrat F J, Meeker T, Gregorio J, Chan J H, Uematsu S, Akira S, et al. Nucleic acids of mammalian origin can act as endogenous ligands for Toll-like receptors and may promote systemic lupus erythematosus. J Exp Med. 2005; 202(8):1131-9.
42. Garcia-Olmo D C, Ruiz-Piqueras R, and Garcia-Olmo D. Circulating nucleic acids in plasma and serum (CNAPS) and its relation to stem cells and cancer metastasis: state of the issue. Histol Histopathol. 2004; 19(2):575-83.
43. Hajizadeh S, DeGroot J, TeKoppele J M, Tarkowski A, and Collins L V. Extracellular mitochondrial DNA and oxidatively damaged DNA in synovial fluid of patients with rheumatoid arthritis. Arthritis Res Ther. 2003; 5(5):R234-40.
44. Sozzi G, Conte D, Leon M, Ciricione R, Roz L, Ratcliffe C, et al. Quantification of free circulating DNA as a diagnostic marker in lung cancer. J Clin Oncol. 2003; 21(21):3902-8.
45. Stieger P, Daniel J M, Tholen C, Dutzmann J, Knopp K, Gunduz D, et al. Targeting of Extracellular RNA Reduces Edema Formation and Infarct Size and Improves Survival After Myocardial Infarction in Mice. J Am Heart Assoc. 2017; 6(6).
46. Gauthier V J, Tyler L N, and Mannik M. Blood clearance kinetics and liver uptake of mononucleosomes in mice. J Immunol. 1996; 156(3):1151-6.
47. Chen W, Janz D R, Shaver C M, Bernard G R, Bastarache J A, and Ware L B. Clinical Characteristics and Outcomes Are Similar in ARDS Diagnosed by Oxygen Saturation/Fio2 Ratio Compared With Pao2/Fio2 Ratio. Chest. 2015; 148(6):1477-83.
48. Gao W, Xiong Y, Li Q, and Yang H. Inhibition of Toll-Like Receptor Signaling as a Promising Therapy for Inflammatory Diseases: A Journey from Molecular to Nano Therapeutics. Front Physiol. 2017; 8:508.
49. Gay N J, Symmons M F, Gangloff M, and Bryant C E. Assembly and localization of Toll-like receptor signalling complexes. Nat Rev Immunol. 2014; 14(8):546-58.
50. Lawton J A, and Ghosh P. Novel therapeutic strategies based on toll-like receptor signaling. Curr Opin Chem Biol. 2003; 7(4):446-51.
51. Yanai H, Ban T, Wang Z, Choi M K, Kawamura T, Negishi H, et al. HMGB proteins function as universal sentinels for nucleic-acid-mediated innate immune responses. Nature. 2009; 462(7269):99-103.
52. Yanai H, and Taniguchi T. Nucleic acid sensing and beyond: virtues and vices of high-mobility group box 1. J Intern Med. 2014; 276(5):444-53.
53. Ogawa E N, Ishizaka A, Tasaka S, Koh H, Ueno H, Amaya F, et al. Contribution of high-mobility group box-1 to the development of ventilator-induced lung injury. American journal of respiratory and critical care medicine. 2006; 174(4):400-7.
54. Abraham E, Arcaroli J, Carmody A, Wang H, and Tracey K J. HMG-1 as a mediator of acute lung inflammation. J Immunol. 2000; 165(6):2950-4.
55. Wolfson R K, Chiang E T, and Garcia J G. HMGB1 induces human lung endothelial cell cytoskeletal rearrangement and barrier disruption. Microvasc Res. 2011; 81(2): 189-97.
56. Fischer S, Gerriets T, Wessels C, Walberer M, Kostin S, Stolz E, et al. Extracellular RNA mediates endothelial-cell permeability via vascular endothelial growth factor. Blood. 2007; 10(7):2457-65.
57. D'Alessio F R, Craig J M, Singer B D, Files D C, Mock J R, Garibaldi B T, et al. Enhanced resolution of experimental ARDS through IL-4-mediated lung macrophage reprogramming. Am J Physiol Lung Cell Mol Physiol. 2016; 310(8):L733-46.
58. Gao X, Anderson D R, Brown A W, Lin H, Amnuaysirikul J, Chua A L, et al. Pathological studies on the protective effect of a macrolide antibiotic, roxithromycin, against sulfur mustard inhalation toxicity in a rat model. Toxicol Pathol. 2011; 39(7): 1056-64.
59. McElroy C S, Min E, Huang J, Loader J E, Hendry-Hofer T B, Garlick R B, et al. From the Cover: Catalytic Antioxidant Rescue of Inhaled Sulfur Mustard Toxicity. Toxicol Sci. 2016; 154(2):341-53.
60. Hassan Z M, Ebtekar M, Ghanei M, Taghikhani M, Noori Daloii M R, and Ghazanfari T. Immunobiological consequences of sulfur mustard contamination. Iran J Allergy Asthma Immunol. 2006; 5(3): 101-8.
61. Vijayaraghavan R, Kulkarni A, Pant S C, Kumar P, Rao P V, Gupta N, et al. Differential toxicity of sulfur mustard administered through percutaneous, subcutaneous, and oral routes. Toxicology and applied pharmacology. 2005; 202(2):180-8.
62. Weiland J E, Davis W B, Holter J F, Mohammed J R, Dorinsky P M, and Gadek J E. Lung neutrophils in the adult respiratory distress syndrome. Clinical and pathophysiologic significance. Am Rev Respir Dis. 1986; 133(2):218-25.
63. Collin A, Lebreton Y, Fillaut M, Vincent A, Thomas F, and Herpin P. Effects of exposure to high temperature and feeding level on regional blood flow and oxidative capacity of tissues in piglets. Exp Physiol. 2001; 86(1):83-91.
64. Anderson D R, Byers S L, and Vesely K R. Treatment of sulfur mustard (HD)-induced lung injury. J Appl Toxicol. 2000; 20 Suppl 1:S129-32.
65. McClintock S D, Till G O, Smith M G, and Ward P A. Protection from half-mustard-gas-induced acute lung injury in the rat. J Appl Toxicol. 2002; 22(4):257-62.
66. Campbell E L, Bruyninckx W J, Kelly C J, Glover L E, McNamee E N, Bowers B E, et al. Transmigrating neutrophils shape the mucosal microenvironment through localized oxygen depletion to influence resolution of inflammation. Immunity. 2014; 40(1):66-77.
67. Zemans R L, Briones N, Campbell M, McClendon J, Young S K, Suzuki T, et al. Neutrophil transmigration triggers repair of the lung epithelium via beta-catenin signaling. Proc Natl Acad Sci USA. 2011; 108(38):15990-5.
68. Neudecker V, Brodsky K S, Clambey E T, Schmidt E P, Packard T A, Davenport B, et al. Neutrophil transfer of miR-223 to lung epithelial cells dampens acute lung injury in mice. Sci Transl Med. 2017; 9(408).
69. Gunther A, Mosavi P, Heinemann S, Ruppert C, Muth H, Markart P, et al. Alveolar fibrin formation caused by enhanced procoagulant and depressed fibrinolytic capacities in severe pneumonia. Comparison with the acute respiratory distress syndrome. American journal of respiratory and critical care medicine. 2000; 161(2 Pt 1):454-62.
70. Zouaoui Boudjeltia K, Piagnerelli M, Brohee D, Guillaume M, Cauchie P, Vincent J L, et al. Relationship between CRP and hypofibrinolysis: Is this a possible mechanism to explain the association between CRP and outcome in critically ill patients? Thromb J. 2004; 2(1):7.
71. MacLaren R, and Stringer K A. Emerging role of anticoagulants and fibrinolytics in the treatment of acute respiratory distress syndrome. Pharmacotherapy. 2007; 27(6):860-73.
72. Shibamiya A, Muhl L, Tannert-Otto S, Preissner K T, and Kanse S M. Nucleic acids potentiate Factor VII-activating protease (FSAP)-mediated cleavage of platelet-derived growth factor-BB and inhibition of vascular smooth muscle cell proliferation. Biochem J. 2007; 404(1):45-50.
73. Nakazawa F, Kannemeier C, Shibamiya A, Song Y, Tzima E, Schubert U, et al. Extracellular RNA is a natural cofactor for the (auto-)activation of Factor VII-activating protease (FSAP). Biochem J. 2005; 385(Pt 3):831-8.
74. Ma L, Shaver C M, Grove B S, Mitchell D B, Wickersham N E, Carnahan R H, et al. Kinetics of lung tissue factor expression and procoagulant activity in bleomycin induced acute lung injury. Clin Transl Med. 2015; 4(1):63.
75. Erjefalt J S, Erjefalt I, Sundler F, and Persson C G. Microcirculation-derived factors in airway epithelial repair in vivo. Microvasc Res. 1994; 48(2):161-78.
76. Tientadakul P, Kongkan C, and Chinswangwatanakul W. Use of an automated coagulation analyzer to perform heparin neutralization with polybrene in blood samples for routine coagulation testing: practical, rapid, and inexpensive. Arch Pathol Lab Med. 2013; 137(11):1641-7.

What is claimed:

1. A method for treating acute respiratory distress (ARDS) in a subject, the method comprises the step of administering to the subject a therapeutically effective amount of a nucleic acid scavenger (NAS).

2. The method of claim 1, wherein the NAS is hexadimethrine bromide.

3. The method of claim 1, wherein the NAS is poly(butyl methacrylate-co-(2-demethylaminoeethyl) methacrylate-co-methyl methacrylate) 1:2:1 (EuE100).

4. The method of claim 1, wherein the NAS is administered as a part of a pharmaceutical composition.

5. The method of claim 1, wherein a combination of NASs is administered to the subject.

6. The method of claim 5, wherein the combination contains hexadimethrine bromide, EuE100, or both hexadimethrine bromide and EuE100.

7. The method of claim 1, wherein the ARDS is caused, at least in part, by bacterial pneumonia, viral pneumonia, sepsis, head injury, chest injury, burns, blood transfusions, near drowning, aspiration of gastric contents, pancreatitis, intravenous drug use, abdominal trauma, or a combination of any of the foregoing.

8. The method of claim 1, wherein the ARDS is caused, at least in part, by exposure of the subject to a chemical agent.

9. The method of claim 8, wherein the chemical agent is a sulfur mustard or chlorine.

10. The method of claim 1, wherein the NAS is administered to the subject intravenously, intraperitoneally, parenterally, intramuscularly or orally.

11. The method of claim 1 further comprising administering to the subject an additional active agent.

12. The method of claim 1, wherein the administration increases oxygen saturation in the subject, decreases arterial blood pH in the subject, decreases PaCO₂ in the subject, increases cSO₂ in the subject, increases PaO₂ in the subject, increases the PaO₂/FiO₂ ratio in the subject, or a combination of any of the foregoing.

13. The method of claim 1, wherein the administration decreases mRNA levels of an inflammatory cytokine, increases mRNA levels of an anti-inflammatory cytokine or a combination of the foregoing.

14. The method of claim 13, wherein the inflammatory cytokine is IL-6, IL1a, CXCL-1, CCL-2, or a combination of any of the foregoing.

15. The method of claim 13, wherein the anti-inflammatory cytokine is IL-4.

16. The method of claim 1, wherein the administration decreases an inflammatory response, decreases activation of the coagulation cascade, decreases pulmonary edema, or a combination of any of the foregoing.

17. A method for decreasing the concentration of circulating extracellular nucleic acids in a subject suffering from or suspected of suffering from acute respiratory distress (ARDS), the method comprising the step of administering to the subject a therapeutically effective amount of a NAS.

18. The method of claim 17, wherein the NAS is hexadimethrine bromide.

19. The method of claim 18, wherein the NAS is poly(butyl methacrylate-co-(2-demethylaminoeethyl) methacrylate-co-methyl methacrylate) 1:2:1 (EuE100).

20. The method of claim 17, wherein the NAS is administered as a part of a pharmaceutical composition.

21. The method of claim 17, wherein a combination of NASs is administered to the subject.

22. The method of claim 21, wherein the combination contains hexadimethrine bromide, EuE100, or both hexadimethrine bromide and EuE100.

23. The method of claim 1, wherein the NAS is administered to the subject intravenously, intraperitoneally, parenterally, intramuscularly or orally.

24. The method of claim 1 further comprising administering to the subject an additional active agent.
